(19) United States
(12) Patent Application Publication
(10) Pub. No.: US 2013/0079776 A1
(43) Pub. Date: Mar. 28, 2013

(54) BONE COMPRESSION SYSTEM

(71) Applicant: Paul Zwirkoski, Pinckney, MI (US)

(72) Inventors: Paul Zwirkoski, Pinckney, MI (US); Christopher B. Stewart, Keswick, VA (US)

(21) Appl. No.: 13/392,785

(22) PCT Filed: Aug. 25, 2010
(86) PCT No.: PCT/US2010/046692

§ 371 Date: Jul. 20, 2012

Related U.S. Application Data
(60) Provisional application No. 61/275,091, filed on Aug. 25, 2009.

(51) Int. Cl. A61B 17/84

(52) U.S. Cl. CPC A61B 17/848
USPC 606/62

(57) ABSTRACT

A discrete sacrificial zinc anode is fabricated from one or more slotted and slatted metal plates. The plates are fixed in a parallel planar configuration using conventional fasteners. One or more electrical connection wires are formed with a looped portion for spacing the anode assembly a predetermined distance from a steel reinforcing member.

### REFERENCE TO RELATED APPLICATIONS

This application claims the benefit of the filing date of U.S. Provisional Application No. 61/275,091, filed Aug. 25, 2009, the entirety of which is incorporated herein by reference.

### FIELD OF USE

Described herein are devices and methods for treating bone and other tissue trauma and/or deformities in humans and animals. In particular, the description relates to devices and methods for utilizing compression devices on surgical pins, screws and bone wire such as, for example, a Kirschner Wire or “K-Wire” to facilitate healing after surgery such as, for example, to repair small bones.

### BACKGROUND

Current treatment for injury or deformation of bones such as small metatarsal bones involve fusion techniques typically utilizing a bone wire such as a “K-wire” inserted through the bone fragments and sometimes secured to an anchor bone. The wire is placed through the fractured or displaced bone segments which are then fixated by anchoring the wire to an adjacent anchor bone in the desired alignment for a duration of time to heal, and in most cases, is removed upon completion of the healing process.

Recently, several devices have been developed that incorporate a component that provides compression to the proximal end of the end bone of the treated bone segments containing a bone wire. This compression helps prevent motion and/or loosening of a fixated fractured bone segment or segments. Combined with the anchoring of the bone wire to an adjacent located bone segment, the compression devices attempt to provide more secure placement and stabilization of fractured bones to aid in healing without loosening or movement of the set bones. These devices are not efficient and/or are complex and bulky making their delivery cumbersome for the user and problematic for the patient during healing.

There is a need for devices and methods that can provide more efficient and effective treatment of bone trauma and/or deformities and in particular, small bone trauma and/or deformities. Devices that have been developed to date have failed to adequately provide a simple structure that is easy to use by surgeons. Disclosed are devices and methods that can substantially improve the treatment of small bone injuries and/or deformities. There exists a clear need for compression devices that can be easily adjusted by surgeons both during and subsequent to a procedure. There also exists a need for devices that can be quickly inserted and/or removed in combination with existing techniques, all of which will provide a more efficient procedure, healing time, and higher rate of success.

### BRIEF SUMMARY

Broadly, described herein are devices and methods allowing for the fixation of small traumatized or deformed bones and/or tissue through the utilization of a compression device in conjunction with an anchorable bone wire, pin, or screw.

Several embodiments of compression devices are disclosed herein that significantly improve existing procedures for repair of damaged bones and tissue such as, for example, small bones. By way of example, the compression devices described below could be utilized in the repair and fusion of phalangical and metatarsal bones for the purpose of surgically correcting hammertoe deformities.

In the various disclosed embodiments a compression device is inserted onto a bone wire, pin or screw, and slides proximally along the wire, pin or screw towards the treated bone fragments until the desired compression location is achieved. Typically, the desired location for placement of a compression device is through the soft tissue located in the distal area of the most distal bone fragment such as, for example, a toe phalange being treated, and abutting up against that bone segment. Once the desired compression is achieved the disclosed devices are then locked into their position to secure and maintain the desired compression. The compression would be maintained over the healing period. In some preferred embodiments, the compression devices are adjustable during the healing period to account for any loss of the desired compression due to any number of variable factors including but not limited to subsidence of the device, reduction in inflammation, or patient activity.

Each of the examples of the various embodiments described herein will be set forth in the detailed description below. One common utilization of the disclosed devices would be for small bones such as phalanges. However, the disclosed compression devices could be utilized in any bone repair scenario where the compression of damaged bone fragments and/or other tissue during healing is desired. In the preferred embodiment, the compression device includes a slidable component having a flexible end with a pair of parallel reliefs along the distal portion of the device's long axis differentiating the distal portion of the device from the more rigid proximal end of the device. Abutting a modified bone wire on the inner wall of the compression device is a pair of inwardly protruding male tabs that slide along the bone wire until a desired compression is achieved and therefore a location along the bone wire is selected. Located towards the outer middle portion of the device a locking clip or ring is provided that, once the desired location of the compression device on the modified bone wire is reached, the locking clip or ring is then be moved distally to a locking position near the distal end of the device. When the locking clip is moved distally on the compression device it axially locks the compression device by squeezing together the parallel reliefs on the compression devices distal end, forcing the inner male segment tabs of the compression device into the female slots of the modified bone wire.

In an alternative embodiment the compression device contains two or more male locking segments that are located on the inner wall of the compression device. The tabs are chamfered on their proximal end so that as the tabs are moved proximally along the bone wire, the tabs are able to move in and out of the female slots on the bone wire. In the preferred embodiment the modified bone wire includes two smooth portions and two portions with female relief slots. The tabs slide along the smooth portion until the desired compression is achieved. The device could then be rotated 90° so the tabs align and mate with the female slots of the modified wire by turning the compression device body with respect to the modified bone wire. The turning of the final locked assembly causes advancement or retraction depending on the direction of rotation and the screw form of the K-Wires' threaded portion.

Another alternative embodiment includes a spring loaded pressure compression device. This design allows the compression device to slide proximally along a standard bone wire until the desired location for compression is achieved at the distal bone segment. Once in this desired position the compression device cannot slide distally on the bone wire unless the user moves the spring tab into a perpendicular orientation with respect to the devices long axis.

In another alternative embodiment, the compression device includes a slidable component that would be delivered into the desired location on a modified bone wire and a second crimping component that includes a C-clip that would be inserted or placed around the slidable component when the slidable component is properly located on the bone wire. The C-clip would lock into recesses located along the modified bone wire locking the compression device into the proper axial position along the bone wire.

In another alternative embodiment, the compression device's components include a spring clip with multiple inwardly facing locking tabs. In this embodiment, the compression device includes a slidable component that would slide down a modified bone wire to the desired location at the distal end of the bone from which the bone wire protrudes. The bone wire contains a series of female slots or dimples down two parallel and symmetric sides of the wire that match with two or more male locking tabs on the spring clip that protrude from the inner wall of the device. The slidable compression device is advanced along the wire until desired compression and then rotated 90° so that the spring clip locking tabs would lock into the desired bone wire slots, preventing any backward directional movement of the compression device thereby maintaining the desired compression.

In another alternative embodiment the compression device includes a two piece “collet” assembly that would be utilized with a modified threaded bone wire. In this embodiment, the outer portion of the device does not rotate as the inner portion rotates on the wire threads. In one alternative, the device is self tapping rather than threaded. In another alternative the outer component rotates and the inner component remains static. This would prevent twisting or torqueing of the soft tissue envelope or the distal bone abutting the compression device.

In another alternative embodiment a single component compression device slides down a modified bone wire that contains multiple female grooves along its axis.

With this design there is a quadrant relief along the bone wires length. Radially adjacent to the quadrant relief there are radial reliefs in an adjacent quadrant of the bone wire. The first quadrant relief, along the bone wires' long axis, allows the compression device to slide along it when the compression device tab and bone wire relief are aligned. The partial radial reliefs allow the user to rotate the compression device 90° when the desired location on the bone wire is achieved, forcing the inward tab to engage the radial reliefs and locking the device in its desired location. The compressive device's tab may be deformable and when the compression device is rotated the tab could self tap into the bone wire reliefs.

The disclosed compression devices might be utilized in the repair of small bones with internal bone wire applications, or could utilized with external fixators. They could be utilized anywhere compression is desired to facilitate healing of bone or tissue trauma and/or deformity.

Further objects and advantages of the devices, systems, and methods of the present disclosure are more fully set forth in the detailed description and accompanying drawings.

### DESCRIPTION OF THE DRAWINGS

Embodiments or variations are now described by way of example with reference to the accompanying drawings.

FIG. 1 shows a preferred embodiment of a compression device on a modified bone wire.

FIGS. 2a-c show several views of a tabbed bone wire compression device.

FIG. 2c shows a cutaway of a tabbed compression device.

FIG. 3a-b show several different perspectives of a tabbed compression device.

FIG. 3c shows a cutaway of a tabbed compression device.

FIGS. 4a-c show an example of a locking clip for use with a tabbed compression device.

FIG. 5a shows a cutaway view of a tabbed compression device on a bone wire with a locking clip in an unlocked position.

FIG. 5b shows a cutaway view of a tabbed compression device on a bone wire with a locking clip in a locked position.

FIGS. 6a-c show various perspectives of a modified bone wire.

FIG. 6d shows a cross sectional view of the partially relieved portion of the K-wire.

FIG. 6e shows a side view of four finger or toe bones prior to surgical intervention.

FIG. 6f shows a side view of four finger or toe bones after surgical preparation of the bone joint surfaces that are to undergo fusion.

FIG. 6g shows the insertion of a k-wire antegrade through the three distal finger or toe bones. The threaded proximal end is residing in one of the two bone anchors.

FIG. 6h shows the threading of the k-wire retrograde across the two bone anchors.

FIG. 6i shows the final assembly of a tabbed compression device on a modified bone wire.

FIG. 7a shows a tabbed compression device on a bone wire in an unlocked position.

FIG. 7b shows a tabbed compression device on a bone wire in a locked position.

FIG. 7d-e shows a couple views of a windowed tab compression device.

FIG. 7f show a cutaway view of a windowed tab compression device.

Fig. g-h shows a couple views of a tabbed compression device without locking features.

FIG. 7i shows a cutaway view of a tabbed compression device without locking features.

FIG. 7j shows an isometric view of a tabbed compression device without locking features.

FIG. 7k shows a side view of a tabbed compression device without locking features.

FIG. 7l shows a cutaway view of a tabbed compression device without locking features.

FIGS. 7m-n show cutaway views of the tabbed compression device without locking features. FIG. 7m is in the un-locked position and 7n is shown in the locked position.

FIG. 7o shows a side view of a single locking tab without a locking feature locked in position along a smooth k-wire.

FIG. 7p shows a cutaway view of a single locking tab without a locking feature locked in position along a smooth k-wire.

FIG. 7q-r show various views of a tabbed compression device with a distal body locking feature.

FIG. 7s shows a cutaway view of a tabbed compression device with a distal body locking feature.

FIG. 7t shows a cross-sectional view of a tabbed compression device with a distal body locking feature.

FIG. 7u-v show a couple side views of a tabbed compression device with another embodiment of a distal body locking feature.

FIG. 7x shows an end view of a tabbed compression device with another embodiment of a distal body locking feature.

FIGS. 8a-c show a helical pathway design of a tabbed compression device.

FIG. 9a shows a cutaway view showing the reliefs in the helical pathway tabbed compression device.

FIG. 9b shows a cutaway view of the internal locking tabs in the tabbed compression device.

FIGS. 10a-b show two views of a clip utilized with the helical tabbed compression device.

FIGS. 11a-b show a cutaway view of a helical tabbed compression device. 11a is unlocked and 11b is locked.

FIGS. 12a-b show a helical tabbed compression device on a bone wire. 12a is unlocked and 12 b is locked.

FIGS. 13a-c show a locking ring utilized for locking the tabbed compression device into position.

FIG. 14a shows the pull ring mechanism for moving the spring clip of the tabbed compression device in an unlocked position.

FIG. 14b shows the pull ring mechanism for moving the spring clip of the tabbed compression device in a locked position.

FIG. 14c-d show cutaway views of the pulling mechanism. FIG. 14c is unlocked and 14d is locked.

FIGS. 15a-b show cutaway views of the locking ring mechanism. FIG. 15a is unlocked and 15b is locked.

FIGS. 16a and b show the pull ring mechanism in a locked, FIG. 16b, and unlocked, FIG. 16a, position on a bone wire.

FIGS. 17a-c show various views of a proximal tab compression device that contains several internal male interlocking tabs.

FIGS. 18a-c show different views of a multiple tab compression device that contains several internal male interlocking tabs.

FIG. 19a shows a cutaway of a multiple tabbed compression device on a bone wire.

FIG. 19b shows an enlarged view, a cutaway of a multiple tabbed compression device.

FIG. 20 shows a multiple segmented tabbed device on a modified bone wire.

FIGS. 21a-c show various views of a spring loaded compression device.

FIG. 22a shows a cutaway of a spring loaded compression device on a bone wire.

FIG. 22b shows, an enlarged view, a cutaway of a spring loaded compression device on a bone wire.

FIG. 23 shows a spring loaded compression device on a bone wire.

FIGS. 24a-c show various views of a crimping compression device.

FIG. 25a shows an ISO view of a crimping compression device.

FIG. 25b shows a view of a clip utilized with a crimping compression device.

FIGS. 26a and b show two cutaway perspectives of a crimping compression device. FIG. 26a is unlocked and 26b is locked.

FIGS. 27a-c show several views of a crimping compression device on a bone wire.

FIG. 28 shows an isometric view of a bone wire with a crimping compression device.

FIGS. 29a-c show various views of a compression device body for use with a spring clip.

FIG. 30a shows an isometric view of a spring clip.

FIG. 30b shows a top view of a spring clip.

FIG. 30c shows a cutaway of a spring clip.

FIG. 31a shows a cutaway of a compression device with spring clip on a bone wire.

FIG. 31b shows an enlarged view of a cutaway of a compression device with spring clip.

FIGS. 32a and b show an alternative multi-part compression device with spring clip.

FIG. 33 shows a compression device with spring clip on a bone wire.

FIG. 33 shows a compression device with spring clip on a bone wire.

FIGS. 34a-c show various views of the inner component of a collet compression device.

FIGS. 35a-c show various views of the external component of a collet compression device.

FIG. 36 a shows a cutaway of a collet compression device on a bone wire.

FIG. 36b shows a cutaway of a collet compression device.

FIG. 37 shows a collet compression device on a bone wire.

FIGS. 38a-c show various views of the outer component of an alternative embodiment of a collet compression device.

FIGS. 39a-c show various views of the inner component of an alternative embodiment of a collet compression device.

FIG. 40a shows a cutaway of an alternative collet compression device on a bone wire.

FIG. 40b shows a cutaway of an alternative collet compression device on a bone wire.

FIG. 41 shows an alternative collet compression device on a bone wire.

FIGS. 42a-c show various views of a quarter turn compression device.

FIG. 43 shows a perspective of a quarter turn compression device on a modified bone wire.

FIGS. 44a and b show cutaways of a quarter turn compression device.

FIGS. 45a and b show a top view of a bone wire utilized with a quarter turn compression device.

FIG. 46 shows a of a quarter turn compression device on a bone wire.

FIG. 47 shows a view of a press fit compression device.

FIG. 48 shows a view of a tinnerman clip compression device.

FIG. 49 shows the use of a disclosed compression device with an external fixator apparatus.

FIG. 50 shows a top view of a disclosed compression device with an external fixator apparatus.

FIG. 51 shows an alternative external fixator device.

FIG. 52 shows a cutaway view of a compression device with an internal seal.

FIG. 53 shows a flexible band with tabs.

### DETAILED DESCRIPTION

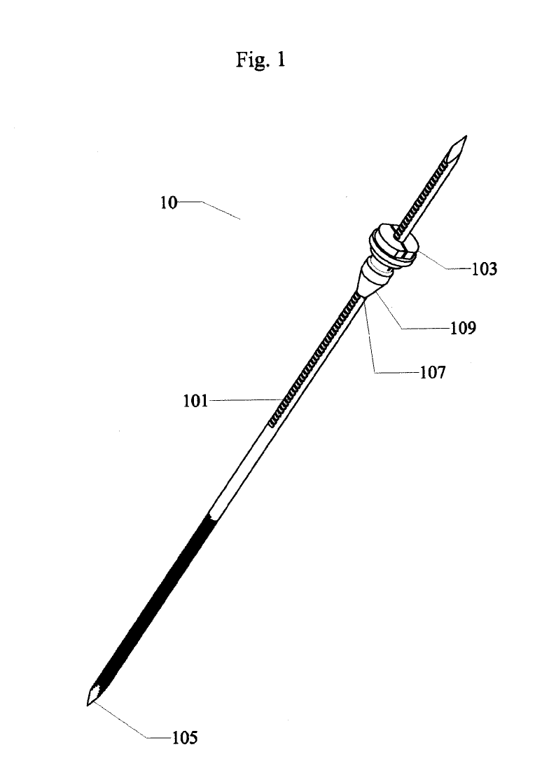

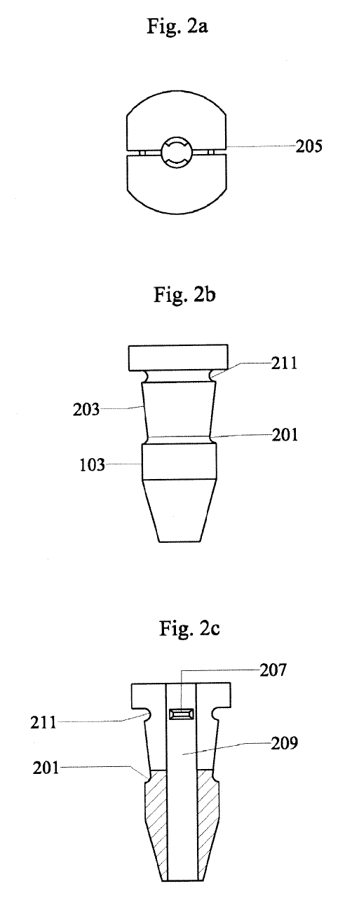

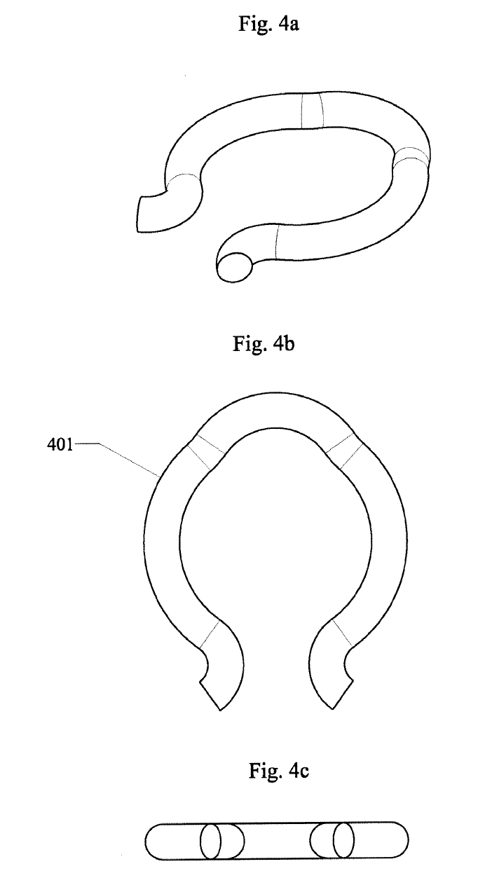

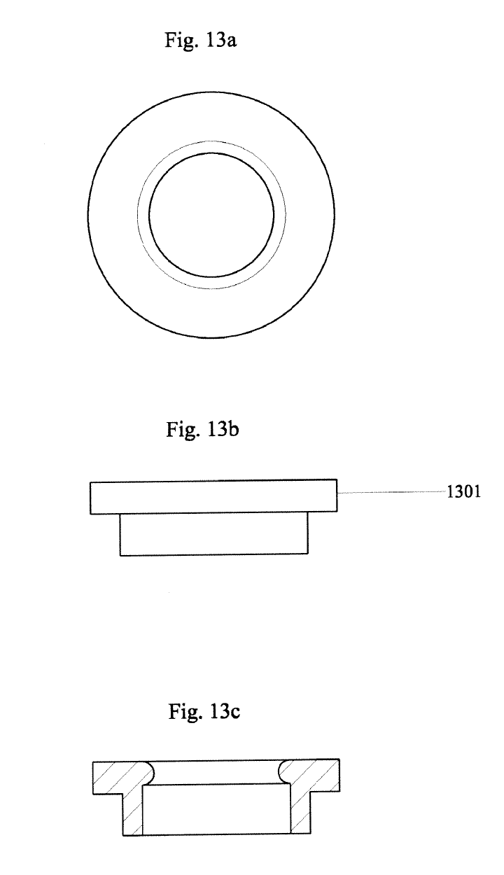

A preferred embodiment of the disclosed bone wire compression apparatus 10 includes a tabbed compression device 103 and a modified bone wire 101 as illustrated in FIG. 1. The compression device includes a tubular-like member that surrounds the modified bone wire and slides axially towards the threaded proximal portion 105 of the modified bone wire 101. The modified bone wire 101 is initially inserted through the various bone fragments that require treatment. Once the desired placement is achieved, the modified bone wire 101 is secured by anchoring the wire by way of threads into a base bone at the threaded proximal end 105 of the modified bone wire 101. When the bone wire anchor is threaded into and secured into the anchor bone the non-threaded distal end of the wire protrudes out from the most distal phalange. The tabbed compression device 103 would be placed on the bone wire and slid proximally on the modified bone wire 101 to the desired location at the distal tip of the most distal phalange. The tapered tip 107 of the tabbed compression device 103 would abut this distal phalange tip. Adjacent to the tapered tip 107 there is a soft tissue spacer 109 to allow the tabbed compression device 103 to have direct bone contact while minimizing trauma to and/or displacement of the surrounding soft tissue. In the disclosed preferred embodiment, as illustrated in FIGS. 2a-c and 3a-c, immediately adjacent and distal to the soft tissue spacer 109, a relief or groove is provided, defining an unlocked-position groove 201 adapted to receive a locking clip, ring, or cap such as, for example, a locking clip 401 as shown in FIGS. 4a-c or a locking ring 1301 as illustrated in FIGS. 13a-13c. When resting in this unlocked-position groove 201, the locking clip 401 or locking ring 1301 would allow the tabbed compression device 103 to freely slide along the modified bone wire 101.

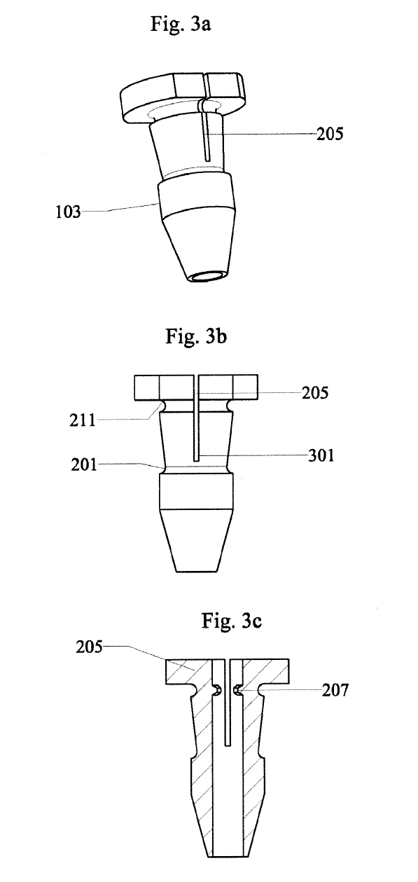

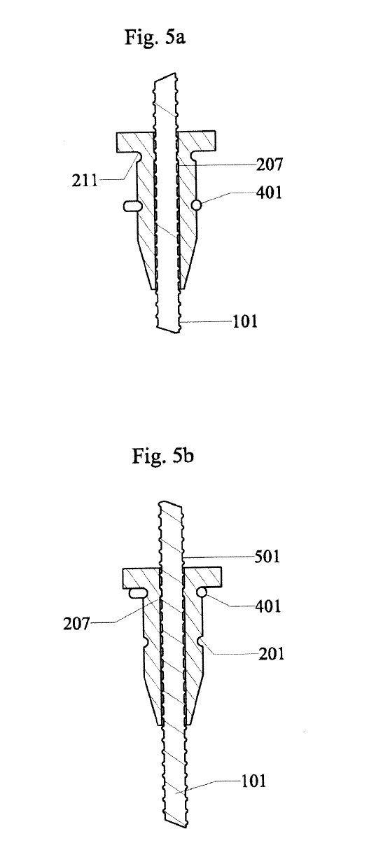

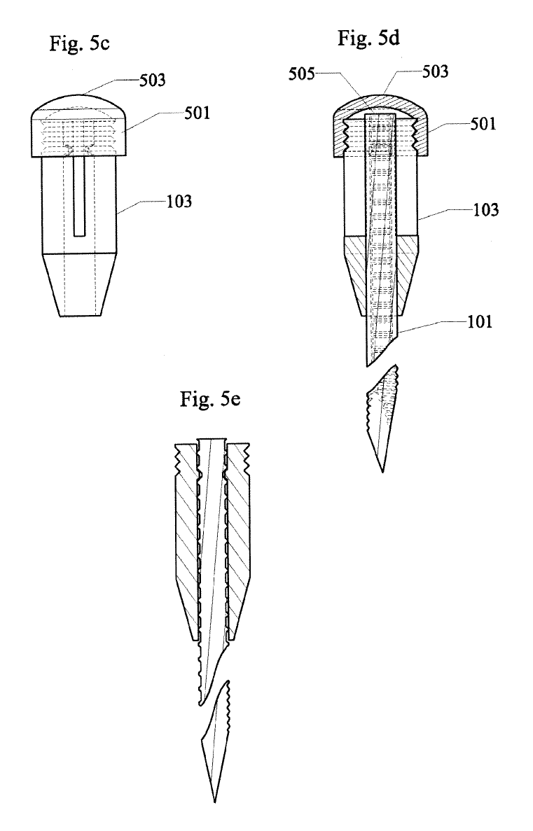

Adjacent and distal to the unlocked-position groove 201 is a locking mechanism ramp 203 and a locked-position groove 211 that facilitate locking the tabbed compression device 103 into place on the modified bone wire 101 once the desired bone compression and location of the tabbed compression device 103 on the modified bone wire 101 is achieved. As depicted in FIGS. 2a-2c and 3a-3c, the locked-position groove 211 and the unlocked-position groove 201 are separate and distinct circumferential grooves spaced axially apart from one another and formed in an exterior, e.g., an outer wall, of the tabbed compression device 103. Moreover, as shown in FIGS. 2b and 2c, for example, the grooves 201, 211 of the present embodiment constitute open channels or troughs formed in the compression device 103 having generally semi-circular cross-sections for receiving the locking clip or ring, for example, in a complementary manner. While the present embodiment, and other embodiments disclosed herein, include both a locked-position groove and an unlocked-position groove, alternative embodiments can include only a locked-position groove. In such embodiments, the locking clip or ring can be stored separate from the compression device 103 while it is being positioned on the bone wire 101, and once positioned as desired, the locking clip or ring can be positioned in the locked-position groove to secure the same. In one preferred embodiment, the tabbed compression device 103 includes two relief cuts 205 that start at a distal end of the tabbed compression device 103 and run parallel to each other ending at a terminus 301 approximately one half to one third of the way proximally down the compression device 103. This splitting of the tabbed compression device 103 created by the two relief cuts 205 defines the tabbed compression device 103 as including at least one flexible member, which in the present embodiment includes two flexible halves, and permits the non-tapered distal portion of the tabbed compression device 103 to naturally bias outwardly when unlocked or to be squeezed together in a radial direction by the locking clip 401 or ring 1301 upon placement of the locking clip or ring into the locked-position groove 211. As such, the at least one flexible member (e.g., the two flexible halves), can be described as being radially actuable because they move in a radial direction to engage or disengage the bone wire 101. In the present embodiment, the placing of the locking clip or ring into the locked position groove 211 would squeeze flexible halves together across the reliefs 205, thereby radially actuating the halves and forcing at least one male locking tab 207 located on the inside wall 209 of the tabbed compression device 103 into a receptive female relief slots 603 located on the modified bone wire 101. In the present embodiment, the at least one male locking tab 207 of the compression device 103 can include a pair of male locking tabs 207, each tab 207 located on an inside wall of a respective one of the two flexible halves. FIG. 5a illustrates a cutaway of the tabbed compression device 103 with the locking clip 401 in an unlocked position, occupying the unlocked-position groove 201, while FIG. 5b illustrates the clip 401 in a locked position, occupying the locked-position groove 211.

While the foregoing embodiment and a variety of embodiments described below each includes a bone wire 101 having female slots 603 receiving the at least one male locking tab 207, alternative embodiments could use smooth bone wires without female slots 603. In such a configuration, the at least one flexible member of the subject compression device 103 would compress against the smooth bone wire in the inward radial direction with sufficient force to resist undesired movement of the compression device 103 relative to the bone wire.

The compression device may include a cap 501 that can be placed on the distal end of the tabbed compression device 103 after it has been placed in the desired location on the modified bone wire 101 as shown in FIGS. 5 c-e. In one embodiment the cap 501 is screwed onto the distal end of the compression device after it has been placed on the modified bone wire and the distal end of the wire 505 has been severed as shown in FIG. 5f. Any method of securing the cap onto the distal end of the compression device might be utilized however. In one variation the top of the cap 501 might be rounded 503 to create a small space to allow for the distal end of the modified bone wire 505 which may protrude from the distal end of the tabbed compression device 103 after being severed as shown in FIG. 5e. The cap might also be utilized as a locking component by squeezing the male tabs located internally within the compression device into female relief slots 603 on the modified bone wire 101 after placement.

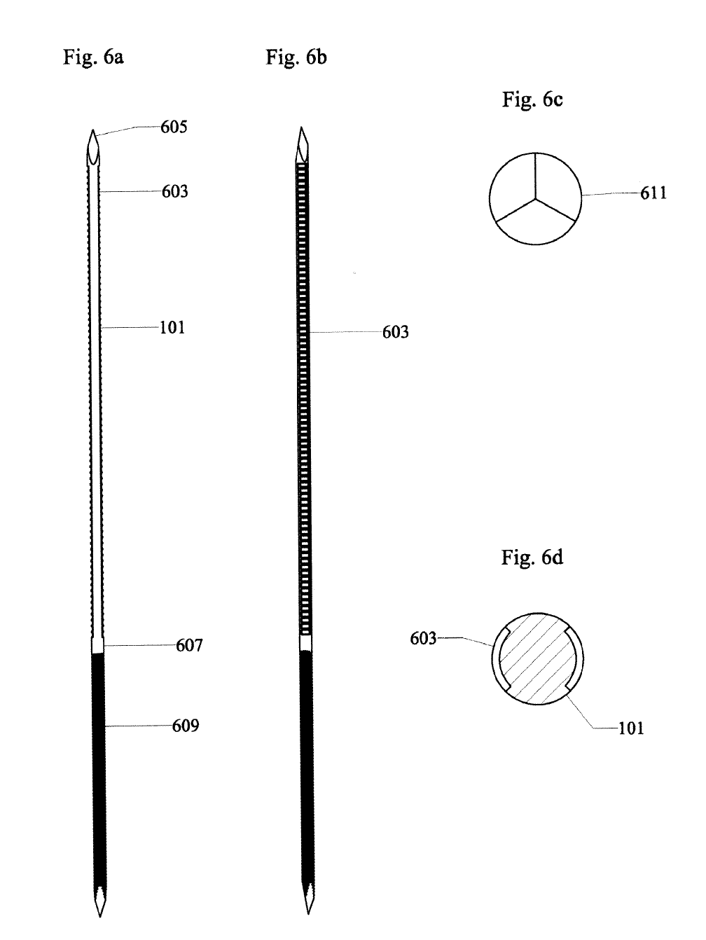

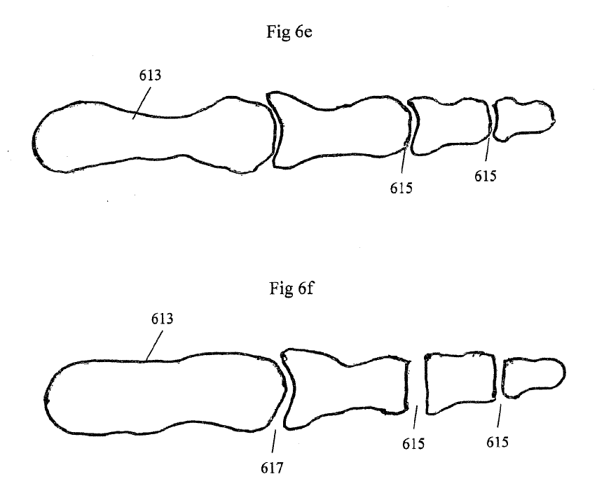

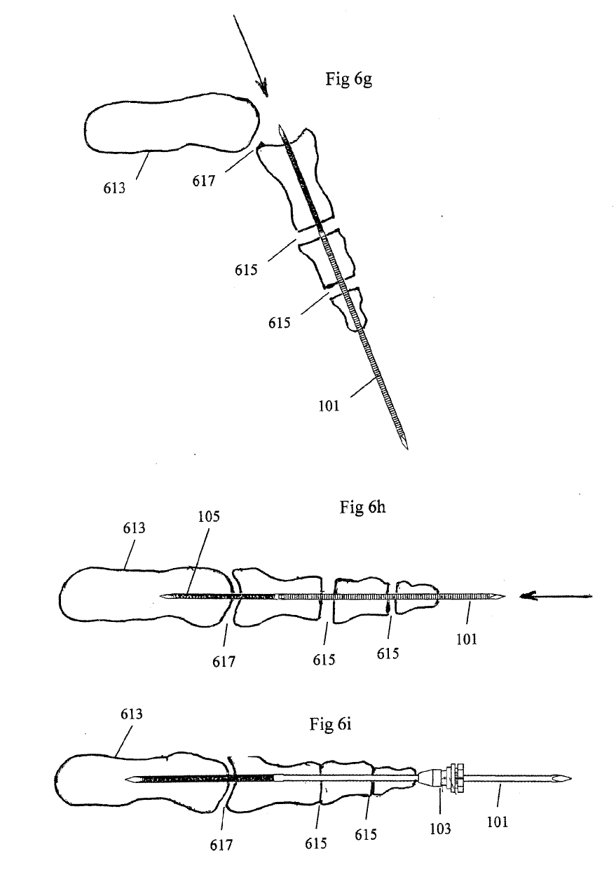

FIGS. 6a and 6b illustrate a modified bone wire 101 for use with a tabbed compression device 103. The modified bone wire 101 has a series of female slots 603 that axially traverse the wire from the distal tip 605 proximally along the modified bone wire 101 to a desired location on the modified bone wire 101. In the disclosed embodiment the female slots cease just distally to bone anchor threads 609. The female relief slots 603 could be placed on the modified bone wire 101 in any location, however, to achieve the locking of the tabbed compression device 103 at the desired location on the modified bone wire 101. As shown in the figures, the female relief slots 603 include separate and distinct depressions, recesses, or cut-outs formed in the modified bone wire 101. From the perspective provided in FIG. 6b, for each female slot 603, the modified bone wire 101 defines opposing upper and lower horizontal planar surfaces and a vertical planar surface that is perpendicular to the upper and lower surfaces. In the disclosed embodiment, the vertical planar surfaces of the slots 603 have generally rectangular profiles, e.g., they are rectangular in shape. That is, when the modified bone wire 101 is viewed from the side in an upright orientation, as shown in FIG. 6b, for example, the female relief slots 603 are rectangular and have a longitudinal dimension that extends transverse to a longitudinal dimension of the bone wire 101. Of course, in other embodiments, the female relief slots 603 could be square, round, oval, or generally any other shape capable of serving the intended purpose. The female relief slots 603 of the present embodiment are not part of a threaded portion of the modified bone wire 101. FIG. 6c illustrates three plane trocar tips 611 located at both tips of a modified bone wire 101 although any number of planes could be utilized to achieve sharpened tips of the modified bone wire 101 FIG. 6d shows a cutaway of the modified bone wire 101 with the symmetric female slots 603.

A method of application is disclosed for utilizing a bone compression apparatus. In this method a modified bone wire is utilized where the proximal portion of a modified bone wire 101 is threaded 609 and the balance of the wire is non-threaded. The threaded portion is provided in order to allow the clinician to pass this portion of the wire across the joint line of a finger or a toe that is not intended to be fused 617 and provide an anchor from which one can utilize the tabbed compression device 103 to afford a compressive force along the axis of the modified bone wire 101 and facilitate fusion of other joint lines 615 provided along the remaining length of the modified bone wire. FIGS. 6 e-i disclose a four step method of utilizing the apparatus to fuse and repair a deformed toe. FIG. 6e shows several joints and bones of a toe. The objective is to fuse the two distal joints 615 and leave the proximal joint 617 free to articulate. The clinician would first prepare the desired joints 615 as shown in FIG. 6f so the bones could abut one another. The modified bone wire 101 is then delivered through the bones and joints 615 to be fused as shown in FIG. 6g. The proximal threaded portion of the bone wire 101 is then inserted into the anchor bone 613 maintaining a separation in the proximal joint 617 as shown in FIG. 6h. The compression device 103 is then placed on the modified bone wire 101 compressing and causing the treated joints 615 to abut each other and fuse.

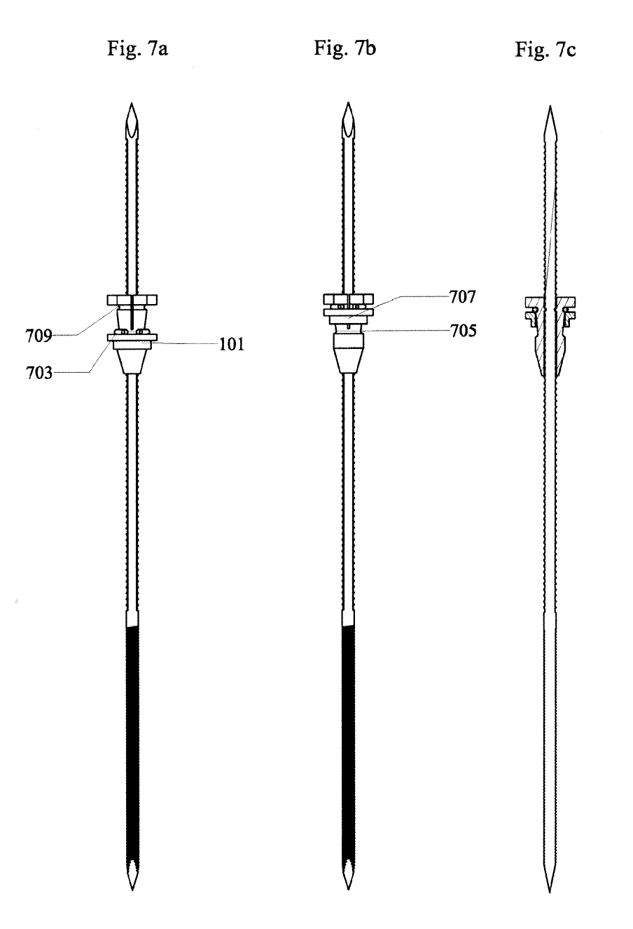

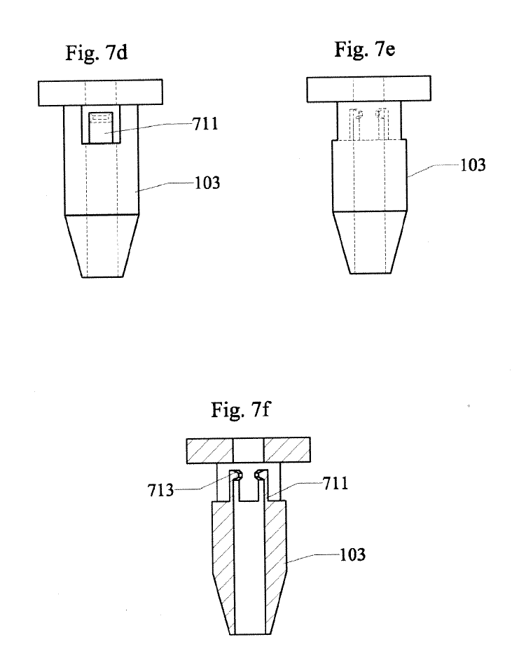

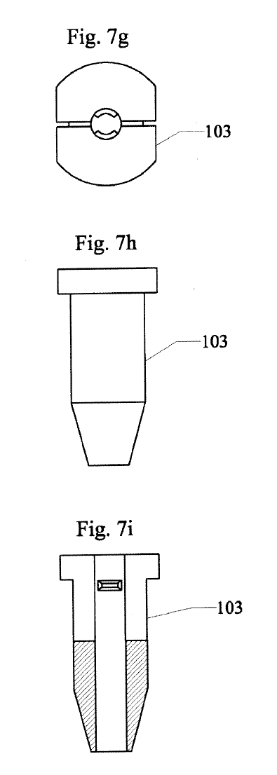

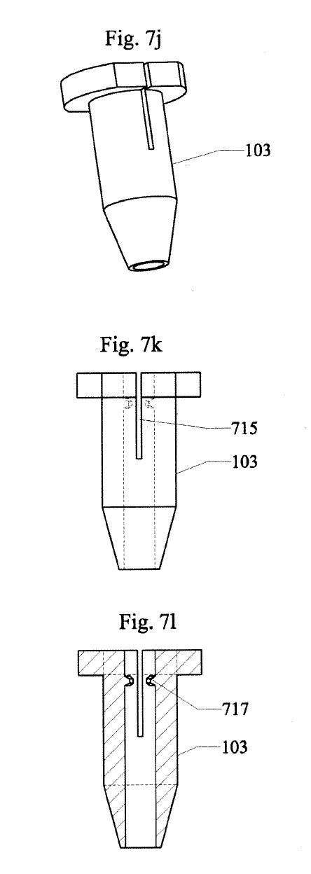

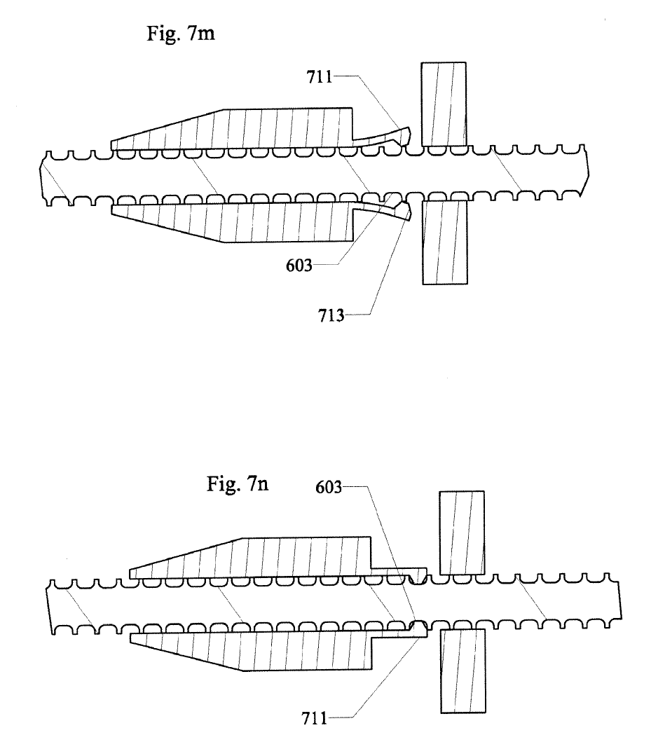

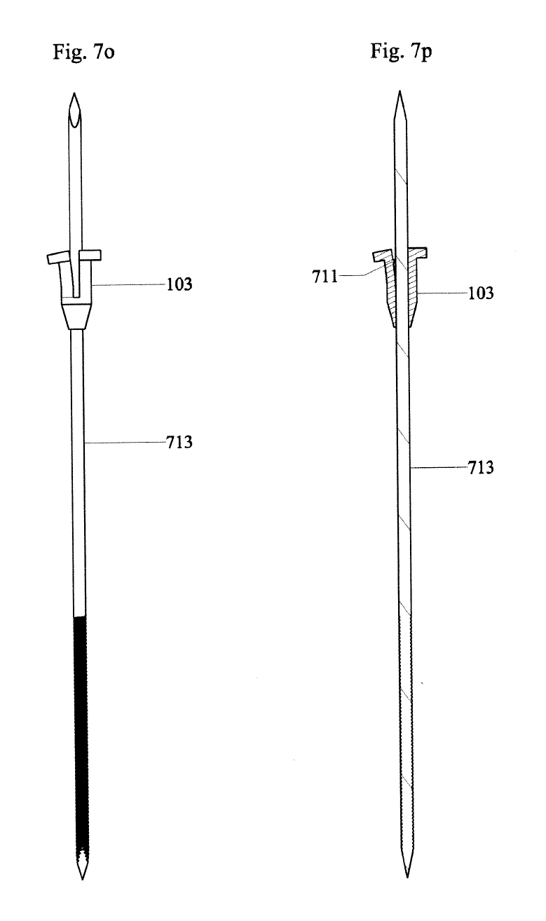

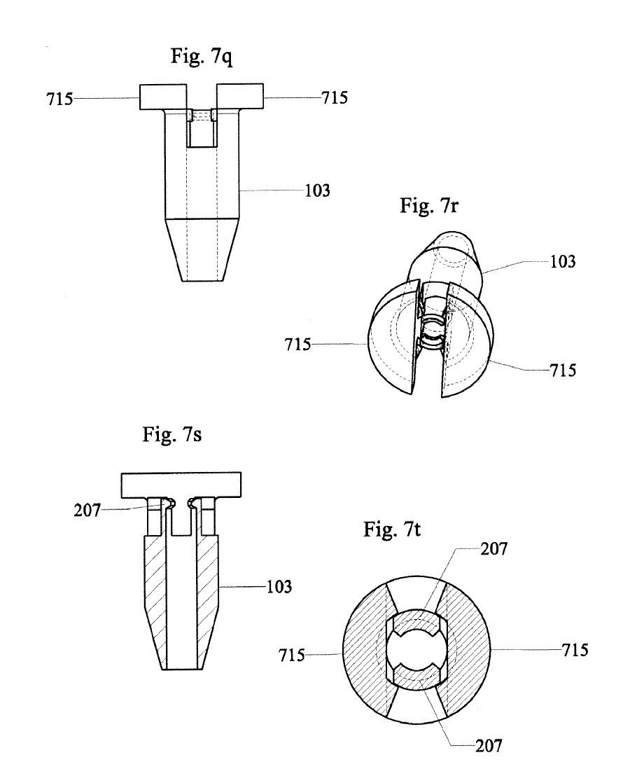

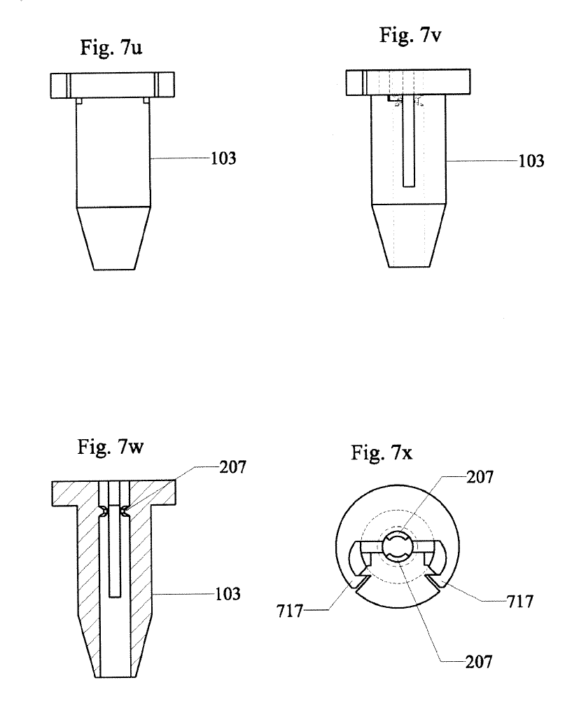

FIG. 7a illustrates the preferred embodiment of the tabbed compression device 103 in an unlocked position while FIG. 7b illustrates it in a locked position. The locking clip 703 is located in the clip resting groove 705 (e.g., unlocked-position groove) where the clip exerts no circumferential load on the flexible body and tabs created by the two relief cuts 205. This allows the tabbed compression device 103 to slide freely down the modified bone wire 101 to the desired location for compression. Once this location is achieved, the locking clip or ring is slid distally on the tabbed compression device 103 towards the locked-position groove 709 where it exerts a radially inwardly-directed circumferential load on flexible halves 708 of the tabbed compression device 103, forcing the male locking tabs 207 located inside the tabbed compression device into the female relief slots 603 of the modified bone wire 101.

FIGS. 7d-n illustrate an alternative embodiment of the tabbed compression device 103 which does not utilize an external locking component. In this embodiment male window tabs 711 might lock into female reliefs 603 at a desired location on the modified bone wire 101 by a rotation into the reliefs. In one embodiment the internal male tabs 711 might snap into the reliefs as the compression device is moved proximally down the modified bone wire through the use of chamfered shaped tabs 713 as shown in FIGS. 7m and n.

In one embodiment as shown in FIGS. 7g-i, the distal portion of the tabbed compression device would not have relief cuts for flexibility. FIG. 7d shows an example of a window tab device where as three sides of the tab are open allowing the tab to be slightly flexible, but able to maintain enough rigidity to lock into the female relief slots 603 without a locking mechanism. As shown in FIGS. 7j-l, in an alternative embodiment the tabbed compression device 103 would contain reliefs 603 and an internal male tab 711 without an external locking component. In one variation the male window tab 711 might be composed of a material of sufficient compressive strength such, as for example, titanium, that the window tab could be deformed enough to expand and subsequently collapse onto a smooth bone wire 713 with sufficient compressive force and friction to prevent axial or rotational movement or sliding along a bone wire. FIGS. 7o and p show a compression device 103 with a single internal male tab 711 compressing onto a smooth bone wire 713 without female relief slots. When the compression device 103 with the single male tab is deformed outwardly from the smooth bone wire 713 it allows the compression device 103 to move along the bone wire to its desired location for optimal bone compression. The single internal male tab would then be collapsed onto the wire maintaining sufficient compressive pressure to lock the compression device into place. While the disclosed example material is titanium any material could be utilized that would provide sufficient compressive strength to lock onto a smooth bone wire 713.

In one variation the compression device 103 might contain internal locking components as shown in FIGS. 7q-t. FIGS. 7p-s show one example of relieved compression device 103 where the opposing sides 715 of the distal end of the compression device can be spread apart allowing the male tabs 207 to slide freely along a modified bone wire to a desired location. When that location is achieved the opposing sides 715 are collapsed inwardly locking the male tabs 207 into place in female relief slots. FIGS. 7u-x show an alternative compression device with an internal locking component. In this embodiment the top distal portion of the compression device 103 includes two deformable and parallel fingers 717 that when in an open position allow the male tabs 207 to move down a modified bone wire to the desired location where the parallel fingers 717 are then closed locking the compression device 103 into the desired position.

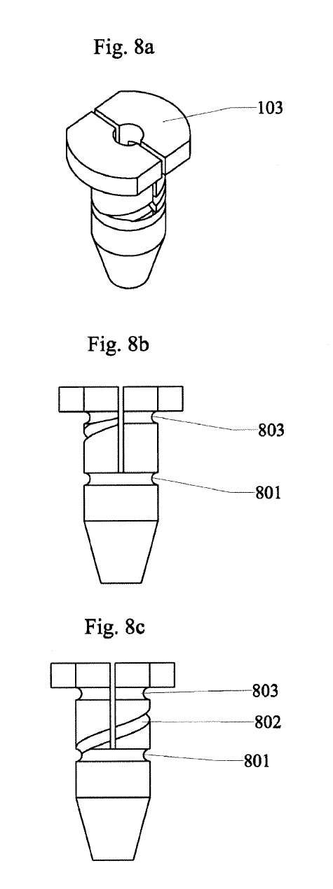

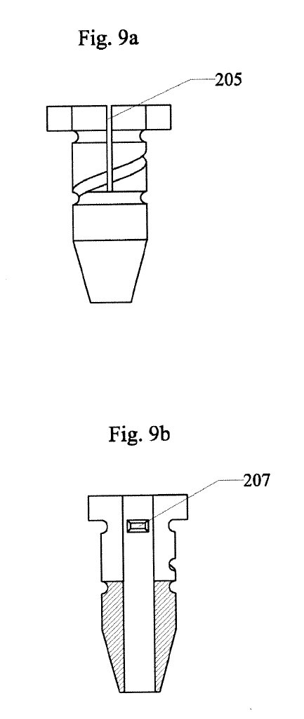

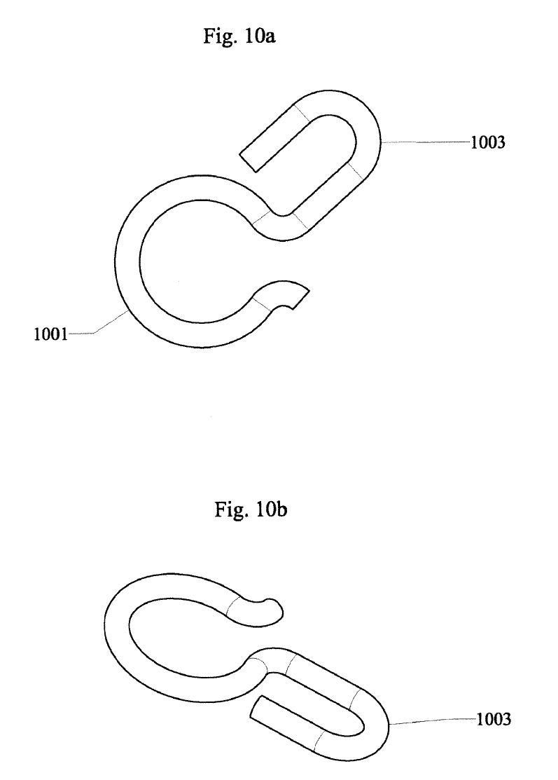

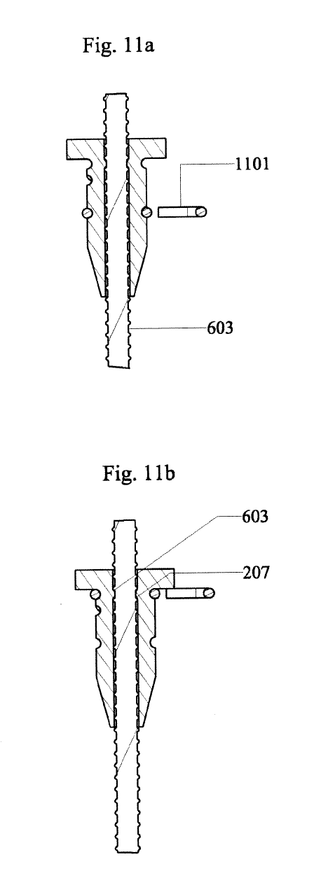

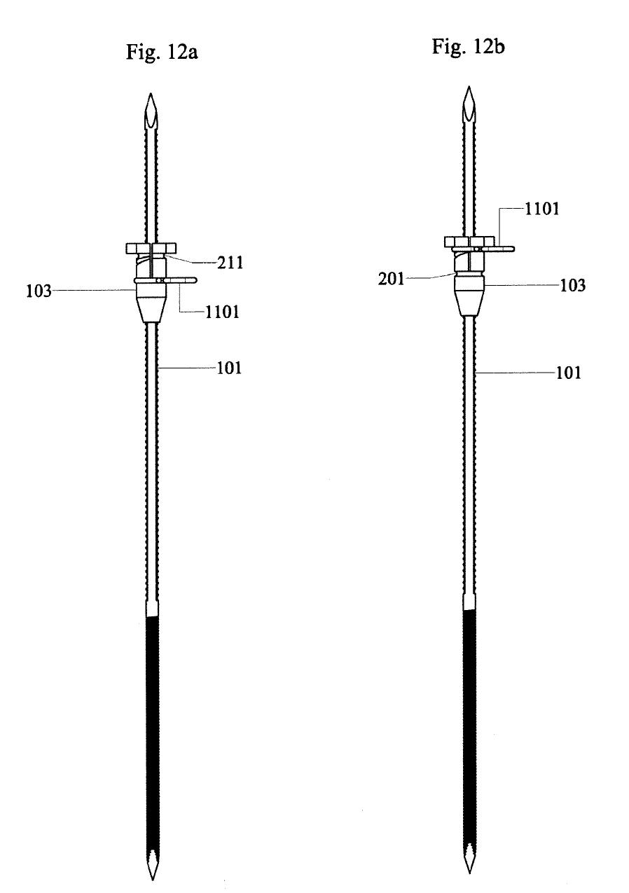

In one alternative embodiment as illustrated in FIGS. 8a-c, the tabbed compression device 103 includes a helical pathway groove 802 extending away from the unlocked position groove 801 around the tabbed compression device 103 and to the locked position groove 803. In this embodiment the locking clip 1001 illustrated in FIGS. 10a and b includes an arm 1003 on the locking clip with arm 1001 or a locking ring that would allow the user to lock the tabbed compression device 103 without pulling the locking clip into the locked position groove as illustrated in FIG. 3, but rather, by rotating the arm 1003 of the locking clip 1001 it would slide distally along the helical pathway groove 802 into a locked-position groove 803. FIG. 9 illustrates a cutaway of the tabbed compression device 103 with the two relief cuts 205 and one of the male locking tabs 207 that is locked into the modified bone wire's 101 female relief slots 603 located in the modified bone wire 101. FIG. 11a illustrates a cutaway of the locking clip with arm 1101 in the unlocked position groove 801 with FIG. 11b illustrating the locking clip with arm 1101 in the locked-position groove 803 with the male locking tab 207 locked into one of the modified bone wire female relief slots 603. FIG. 12a illustrates the tabbed compression device 103 with the locking clip with arm 1101 in an unlocked position groove 201, and FIG. 12b shows the device with the locking clip with arm 1101 in a locked-position groove 211 position on a modified bone wire 101.

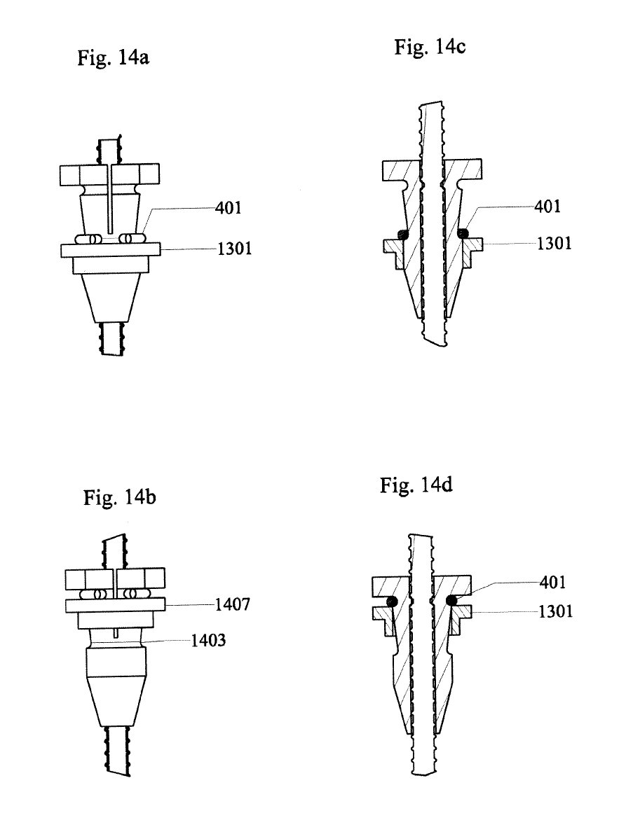

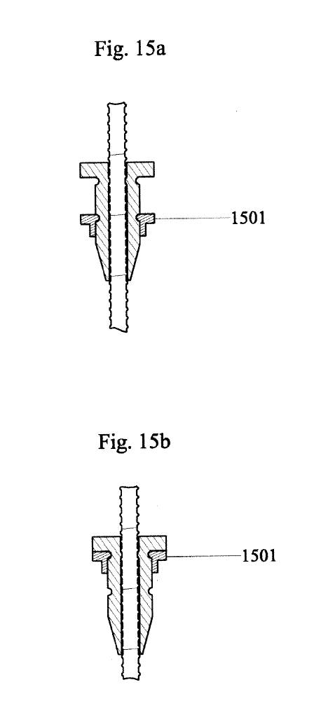

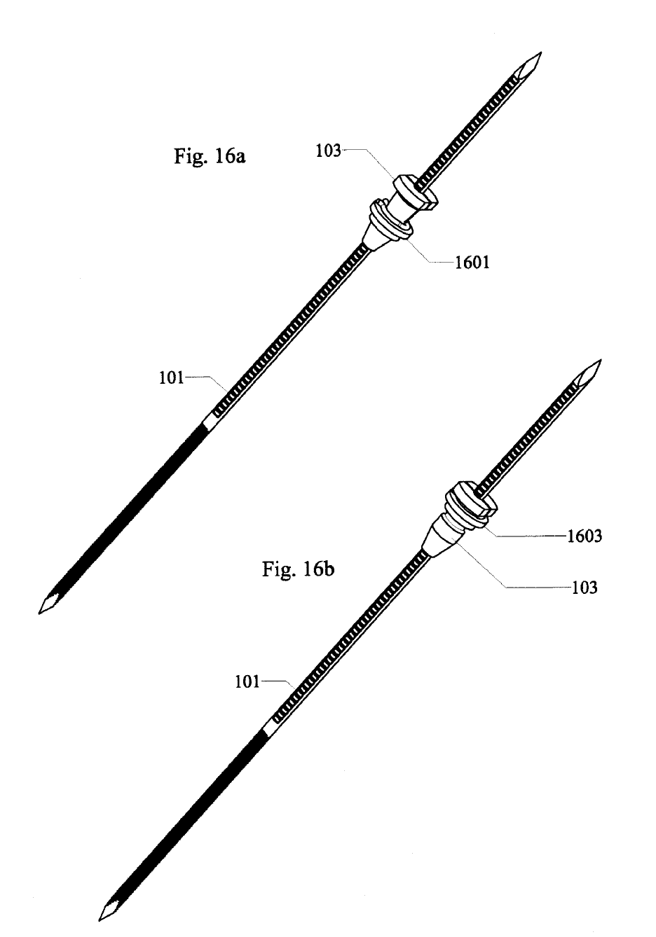

In another alternative embodiment the tabbed compression device 103 includes a pull ring 1301 as illustrated in FIGS. 13a-c. Upon locating the desired location of the tabbed compression device 103 along the modified bone wire 101 the user pulls the pull ring 1301 distally from the unlocked position groove 1403 towards the locked-position groove. This pull ring 1301 either pulls a locking clip or ring 401 into a locked position 1407, as illustrated in FIGS. 14a-d, or the pull ring 1301 is the locking device itself 1501, as illustrated in FIGS. 15a-b. As shown in FIGS. 14a and 14b, the pull ring 1301 includes a cylindrical portion slidably disposed on the tabbed compression device and a pull flange extending radially outward from an outer wall of the cylindrical portion to be grasped by a user to initiate sliding motion. Accordingly, the pull ring 1301 is generally shaped like a top hat and has a cross-section that is generally T-shaped. Additionally, in the embodiment where the pull ring 1301 is the locking clip or ring, as shown in FIGS. 15a-c, the pull ring 1301 further includes a locking ring that extends radially inward from an inner wall of the cylindrical portion of the pull ring 1301, wherein the locking ring is adapted for engagement with the unlocked-position groove (FIG. 15a) and the locked-position groove (FIG. 15b). FIGS. 16a and 16b illustrate the tabbed compression device 103 with a locking ring in an unlocked position 1601 and a locked position 1603.

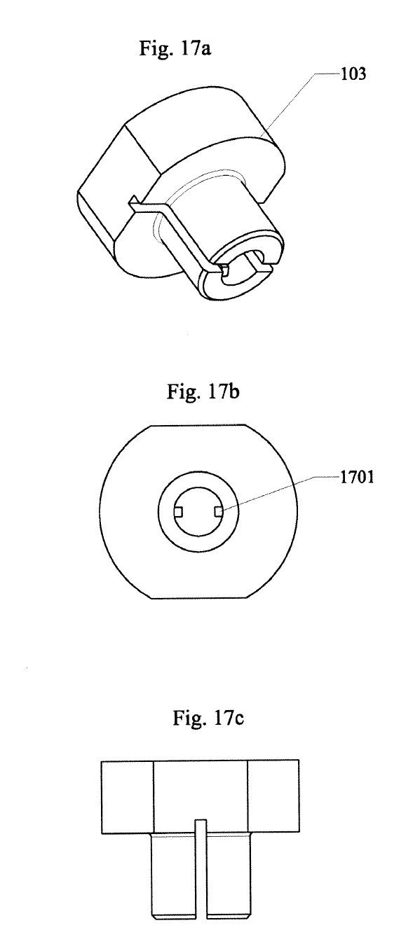

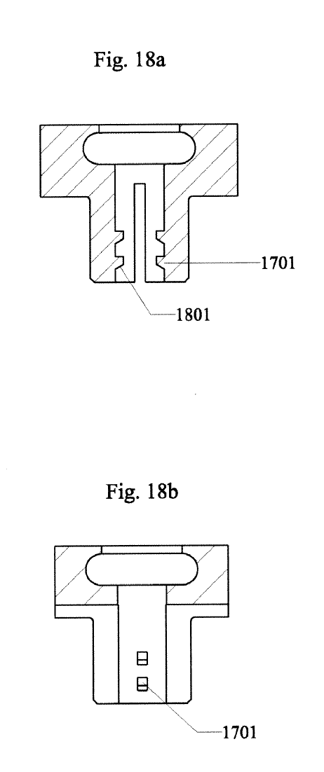

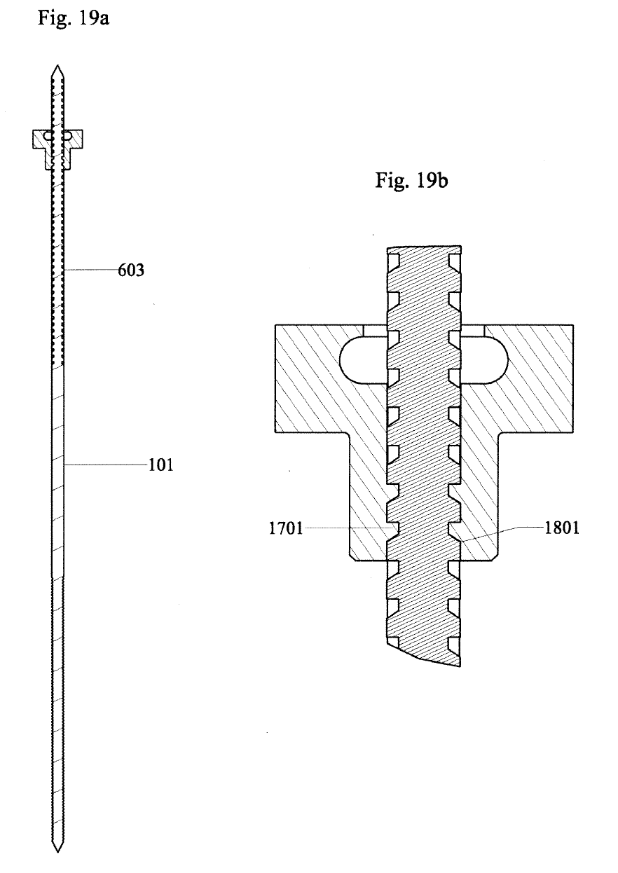

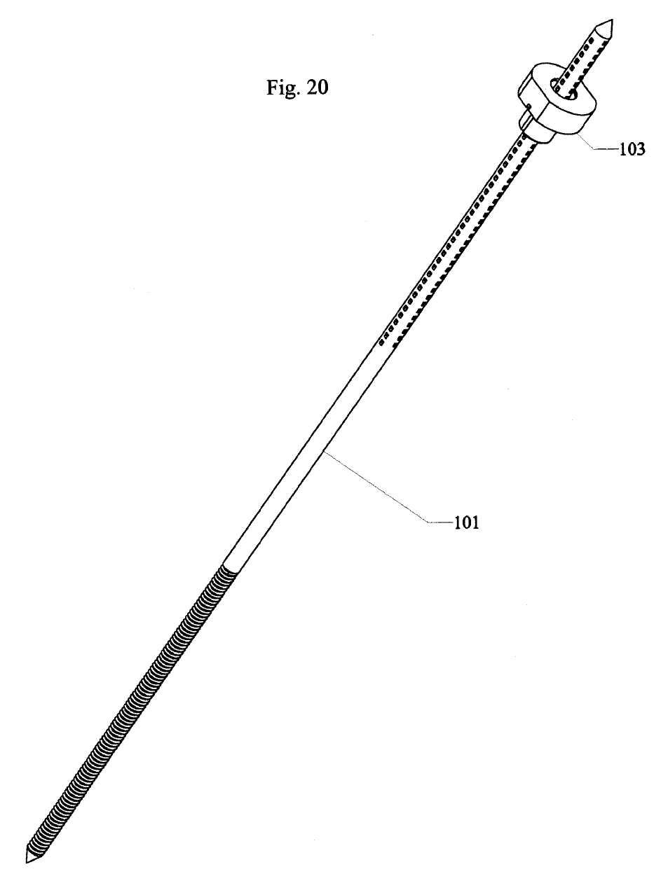

Illustrated in FIGS. 17-20 is an alternative embodiment of the compression device that includes two or more male locking tabs 1701 that are located on the inner distal wall of the compression device 103 and traverse axially along a modified bone wire 101 as illustrated in FIGS. 19a and b. The male locking tabs 1701 of this embodiment are chamfered on one end 1801 so that as they are moved proximally along the bone wire 101, the male locking tabs 1701 are able to move in and out of the female relief slots 603 on the modified bone wire 101 as illustrated in FIGS. 19a and b. Once the desired location is achieved to obtain the desired compression, the male locking tabs 1701 would lock into the female relief slots 603 and would be unable to reverse direction distally on the wire, thus maintaining the desired location and compression. FIG. 20 illustrates the device 103 on a modified bone wire 101. Although the embodiment disclosed in FIGS. 19a-19c does not include a locking clip or ring, the tabbed compression device depicted therein could include a locking clip or ring, with or without a specific locked-position groove and/or an unlocked-position groove.

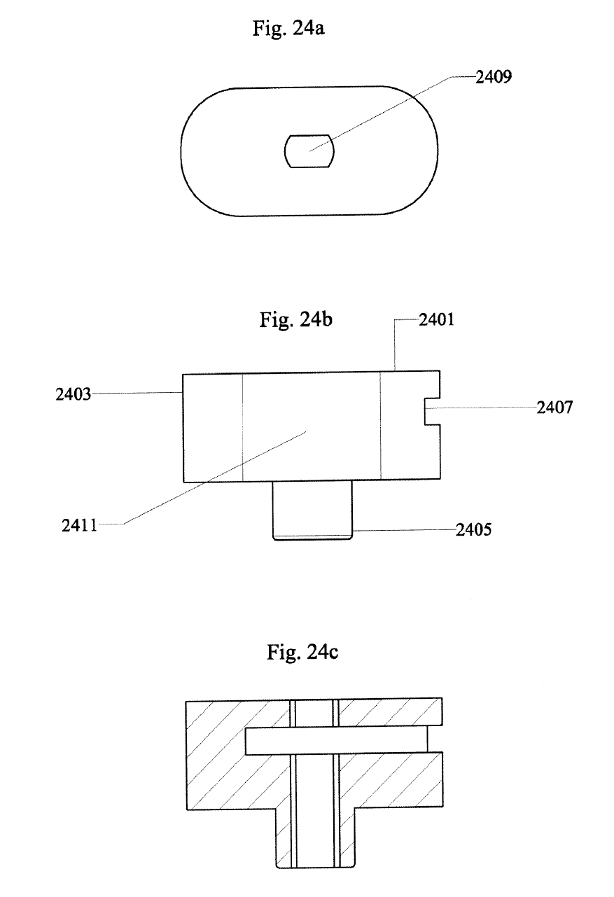

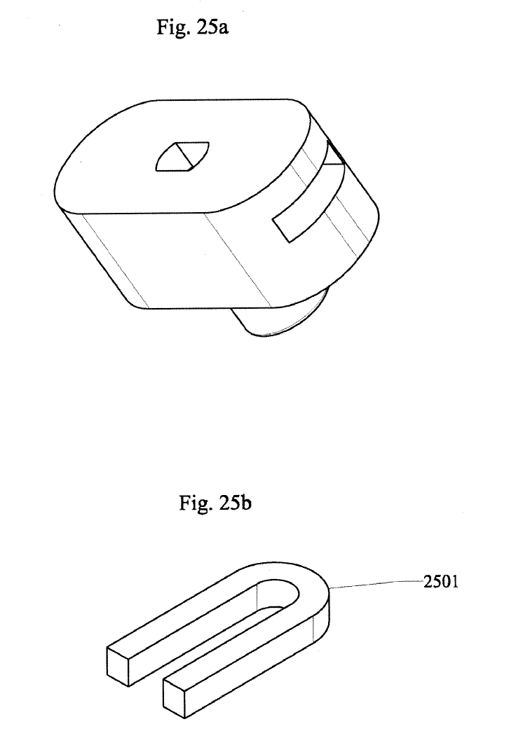

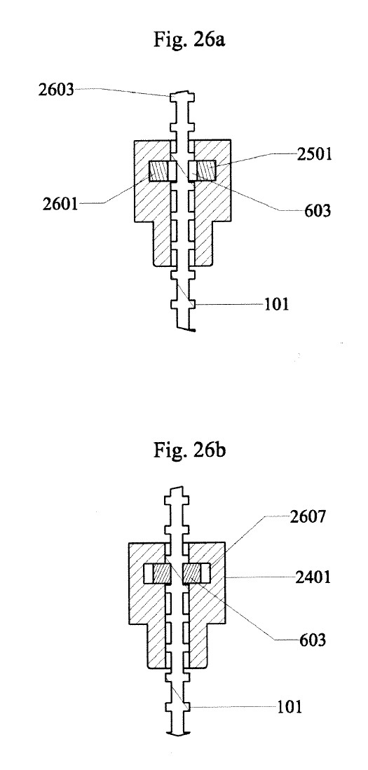

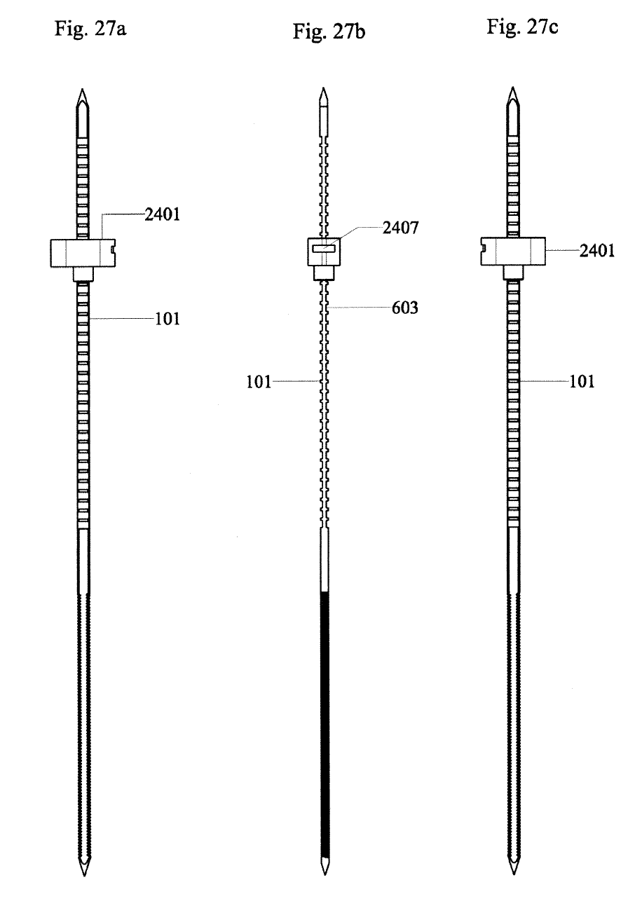

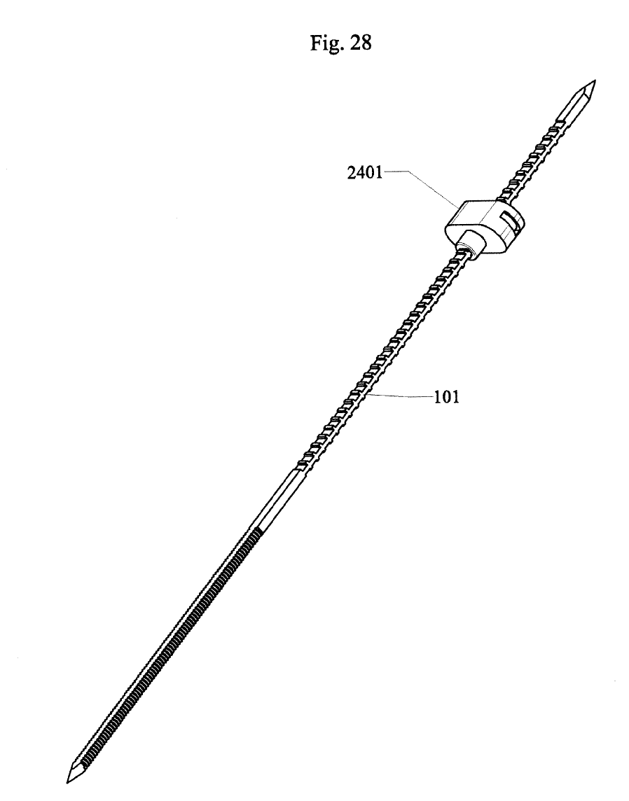

Another alternative embodiment is illustrated in FIGS. 24-28. FIGS. 24a-c illustrates an example of a crimping compression device 2401 that includes a crimping housing component 2403, a soft tissue spacer component 2405, and a receiving slot 2407 for a crimping component 2501 such as illustrated in FIG. 25b. The crimping compression device 2401 slides axially and proximally down a modified bone wire 101 through a bone wire passage 2409 to the desired location abutting the distal phalange bone segment. When the desired compression is achieved, the user might crimp the modified bone wire by squeezing the sides 2411 of the housing component 2403. FIG. 26a illustrates a cutaway with the crimping mechanism 2501 in an unlocked or free position 2601. When the crimping component 2501 is moved from the two sides towards the modified bone wire 101, male tabs 2501 move into the female relief slots 603 on the modified bone wire 101 as shown in FIG. 26b and lock the crimping compression device 2401 into place. The device may be adjustable post procedure by utilizing a removable crimping component 2501 that could be slid into the crimping mechanism receiving slot 2407 in the crimping component 2401, and into the female receptor recesses 603 on the modified bone wire 101 after placement is achieved. FIGS. 27a-c and FIG. 28 illustrate examples of various side and front views of a crimping compression device 2401 on a modified bone wire 101.

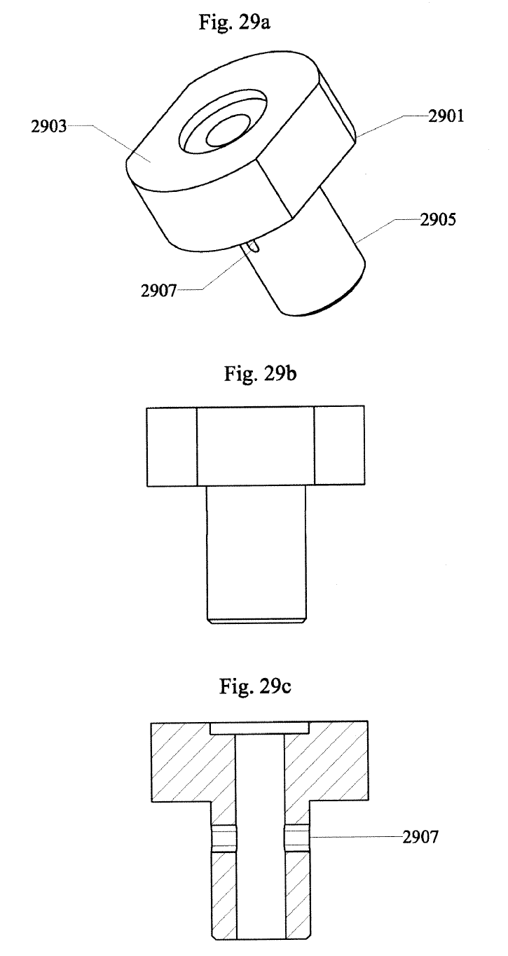

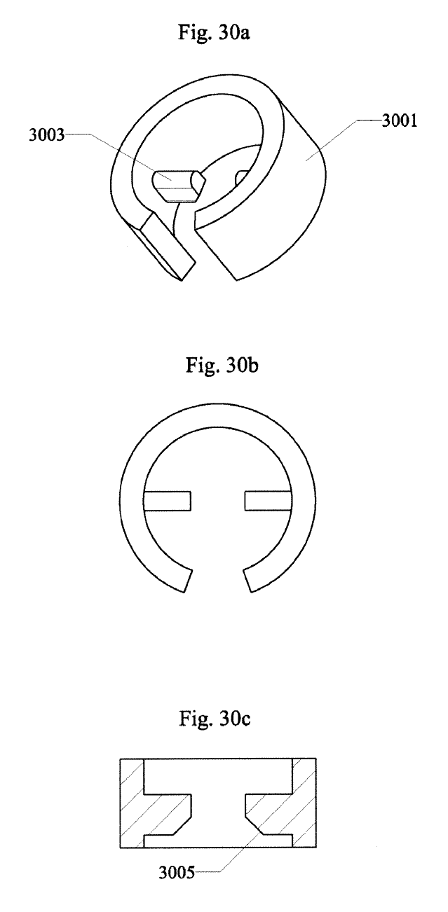

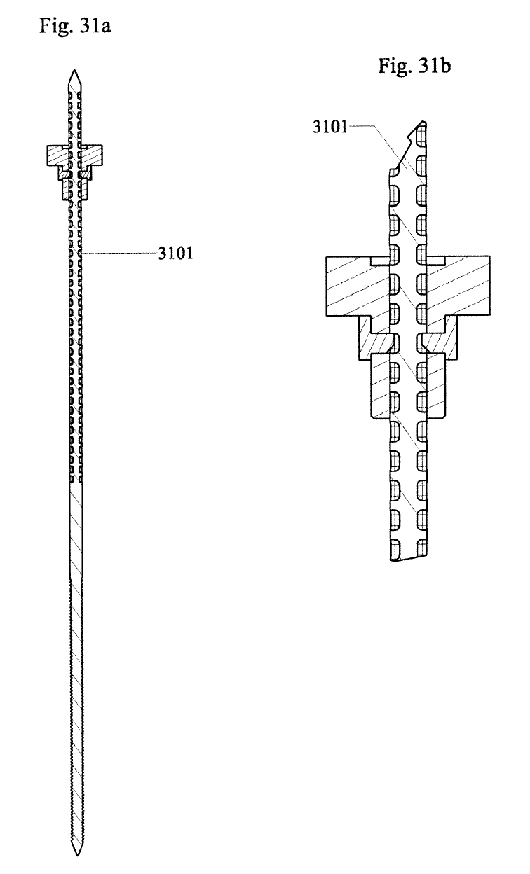

In another alternative embodiment the bone wire compression apparatus 10 includes an alternative spring clip compression device 2901 as illustrated in FIGS. 29-33. This alternative embodiment includes a shoulder component 2903, and a soft tissue component spacer 2905. With this device, a modified locking clip or ring 3001 as illustrated in FIGS. 30 a-c would be placed around the distal portion of the soft tissue spacer 2905 of the spring clip compression device 2901. On the spacer of the spring clip compression device 2901 there are two female receiving slots 2907 coincident to two male locking tabs 3003 disposed on an inside wall of the locking clip or ring 3001 that the tabs could be inserted into. These tabs include slopes or chamfers 3005 that would allow unidirectional movement of the device proximally down a modified bone wire 3101 to the desired location as shown in FIGS. 31a and b.

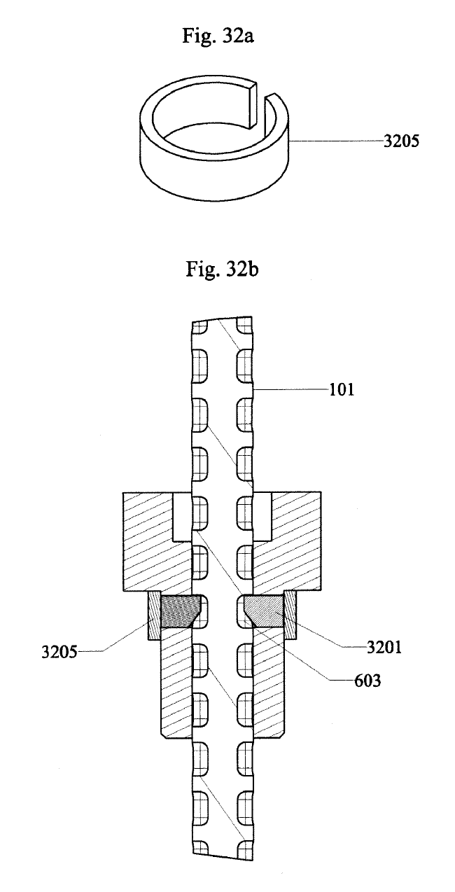

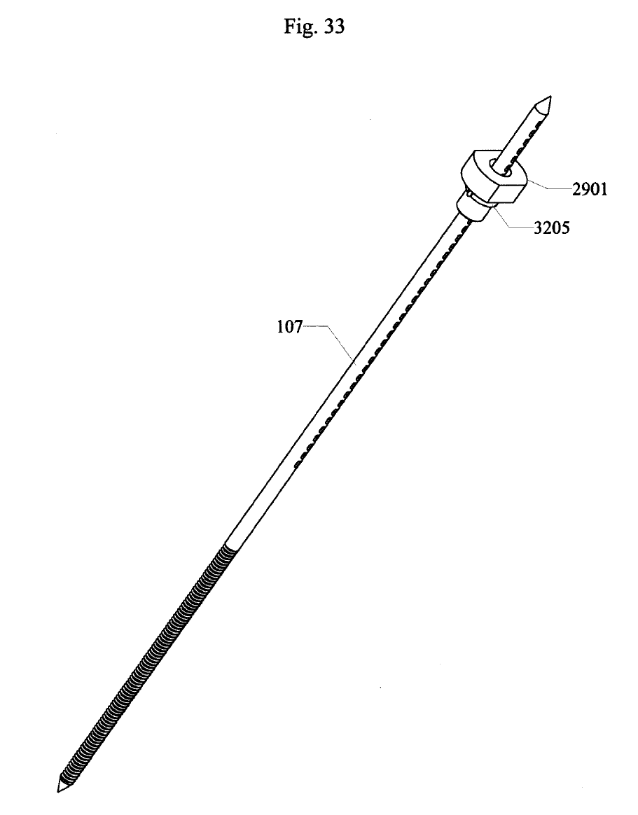

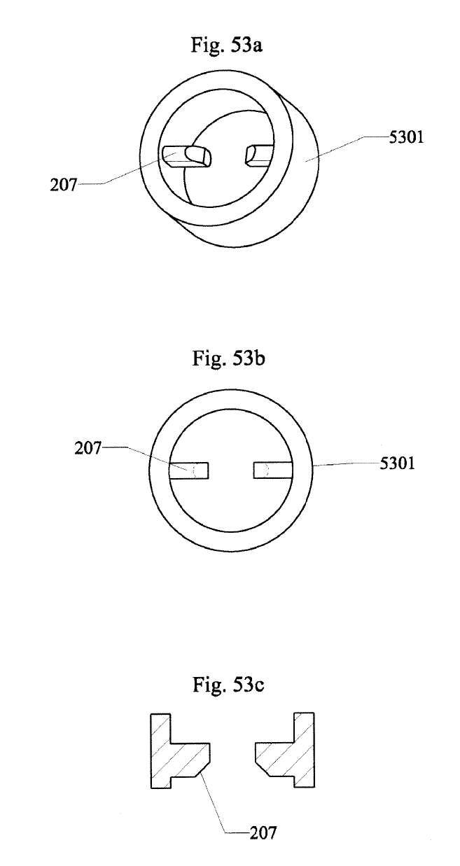

FIGS. 32a and b show an alternative embodiment where separate locking tabs 3201 are placed into the female receiving relief slots 603 and then the locking collar 3205 is placed over metal locking tabs 3201 after the desired location on the modified bone wire 101 is achieved. FIG. 33 shows the spring clip compression device 2901 on a modified bone wire 101 showing the collar 3205 in a locked position. In one variation a flexible band embodiment might be utilized as shown in FIG. 53.

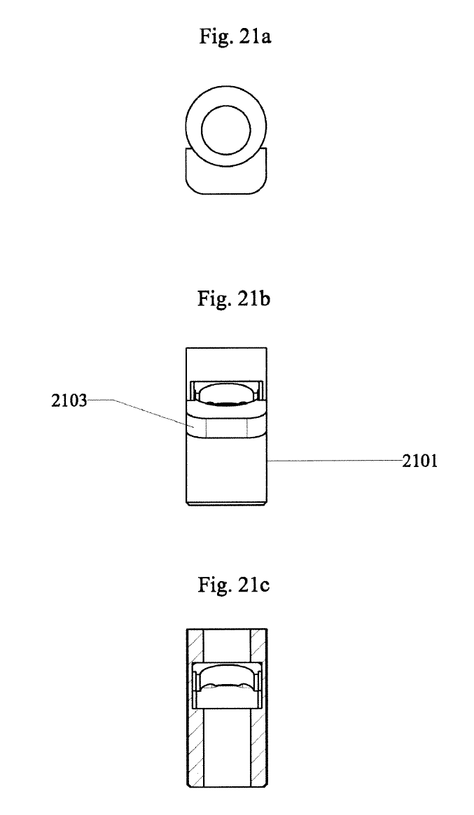

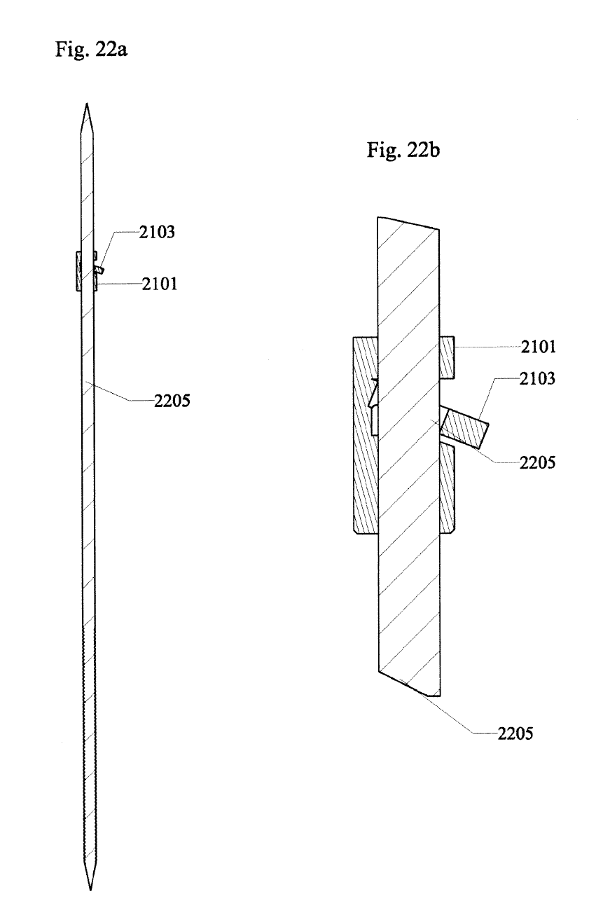

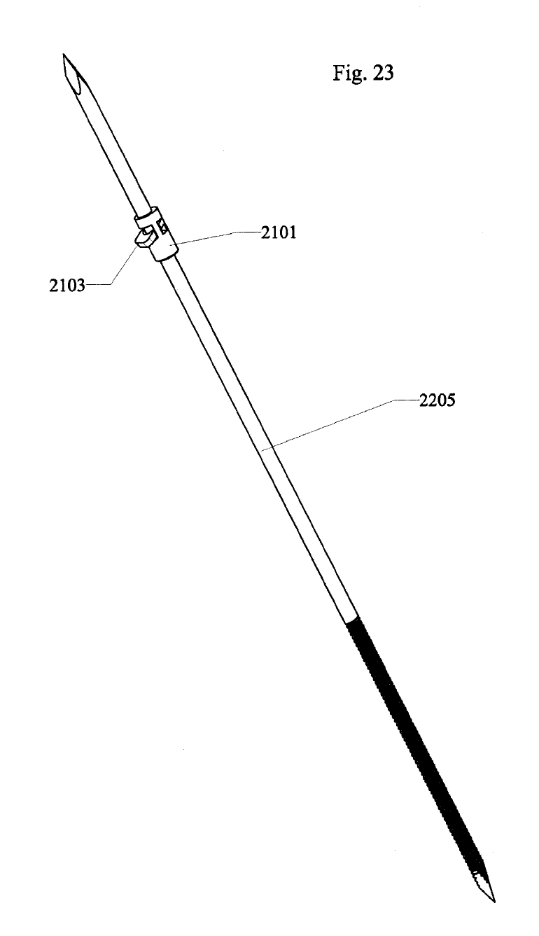

FIGS. 21a-c, 22a-b, and 23 illustrate an alternative spring loaded binding compression device 2101. This design would allow the alternative spring loaded binding compression device 2101 to slide along a modified bone wire 101 until the desired location for compression is achieved. Once in place, the alternative spring loaded binding compression device 2101 could be reversed on the bone wire distally to adjust the compression by moving the spring tab 2103 towards a perpendicular orientation relative to the modified bone wire axis. FIG. 23 illustrates the spring loaded binding compression device 2101 on a modified bone wire 101. The natural spring force of the spring tab 2103 binds with the bone wire 22051 resisting any axial motion in a distal direction.

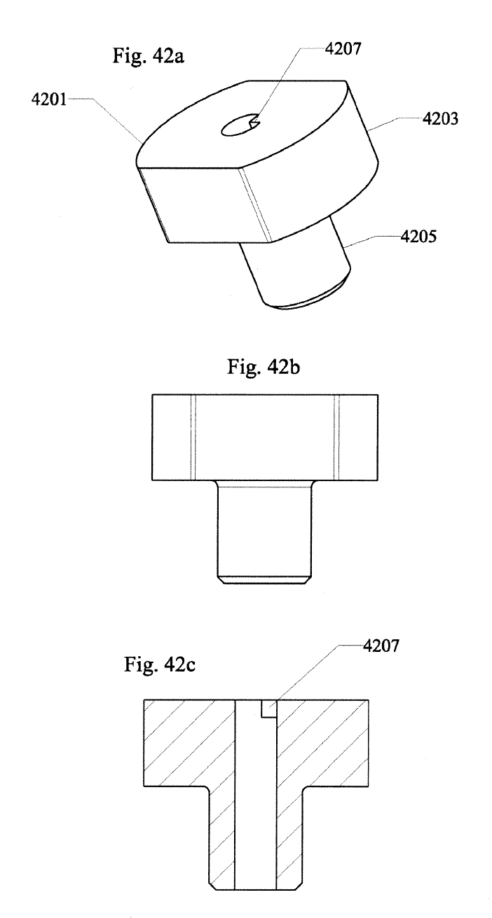

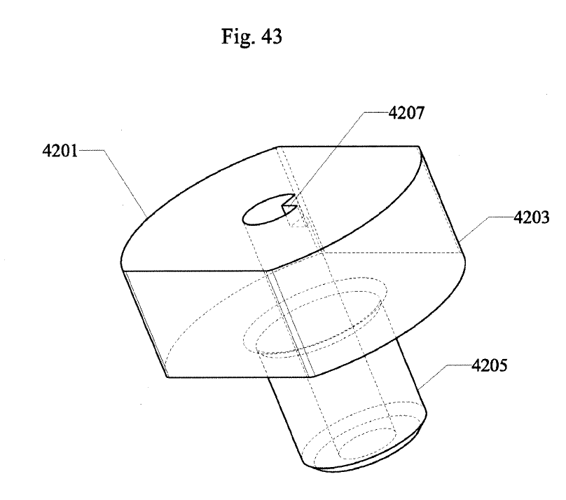

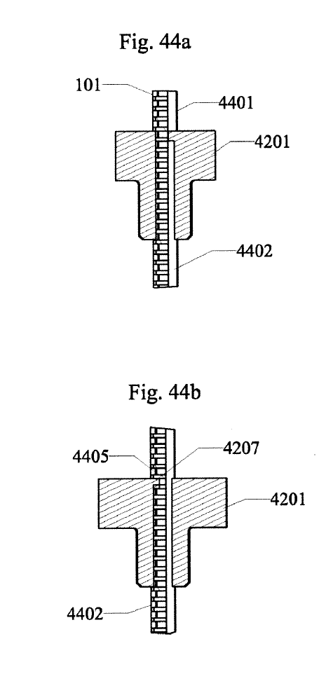

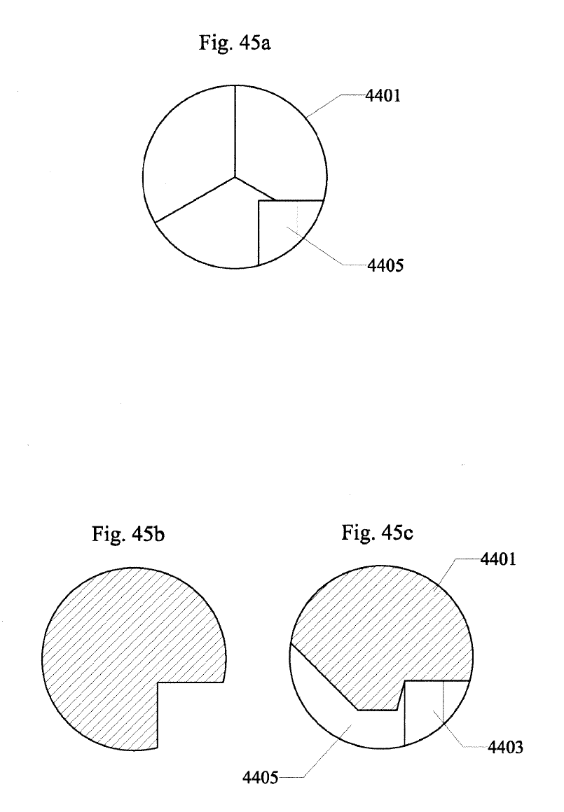

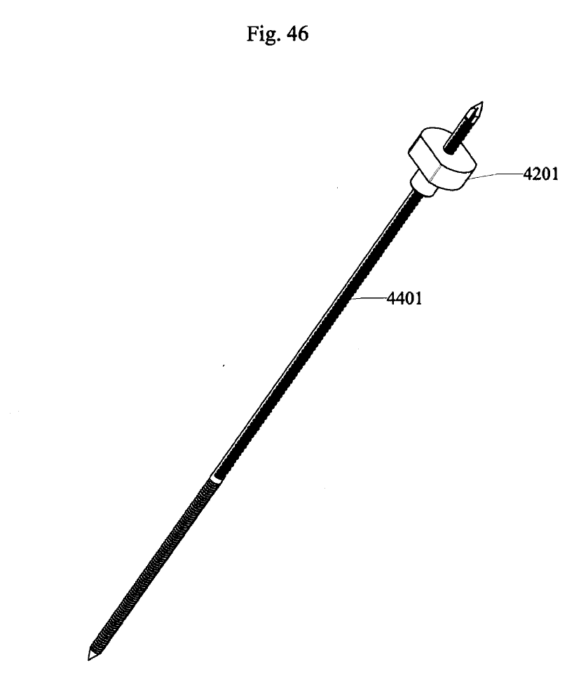

FIGS. 42-46 illustrate another alternative embodiment of a bone wire compression apparatus 10 that allows a quarter turn compression device 4201 to slide proximally down a modified bone wire 101 to the desired location and is then locked into place by rotating the device 90 degrees. This quarter turn compression device 4201 includes a shoulder 4203 and soft tissue spacer 4205. A male tab 4207 is located on the inside wall of the quarter turn compression device 4201 and remains unlocked while in a female receiving track 4403 of the modified bone wire 101. Adjacent to the receiving track would be a series of female receptor slots 4405 that would receive the male locking tab 4207 upon the 90 degree rotation of the compression device locking it into place. FIGS. 45a-c illustrate an end view FIG. 45a and a cutaway FIG. 45b-c of the receiving track 4403 and the receptor slots 4405. FIG. 46 illustrate a completed quarter turn tabbed compression device 4201 on a modified bone wire 4402.

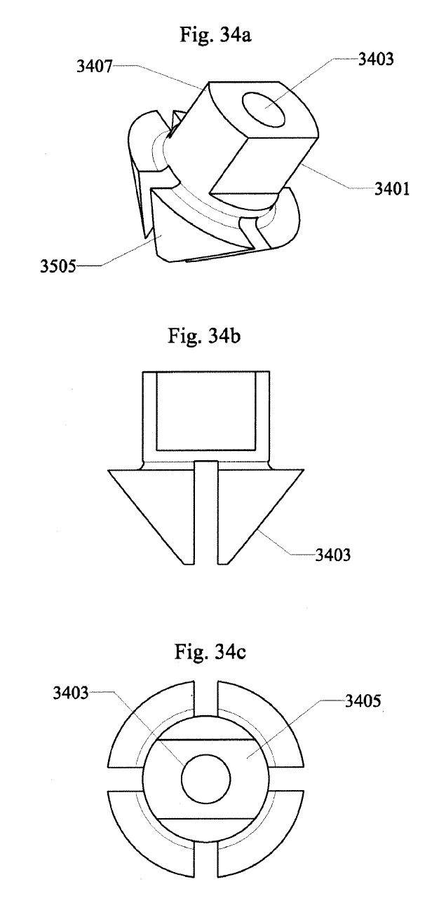

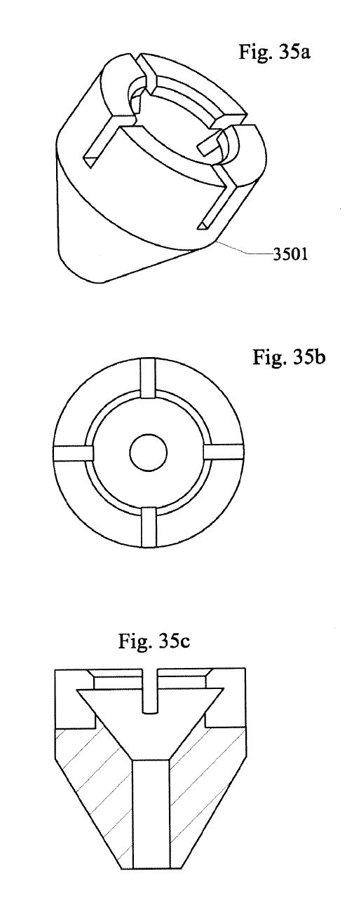

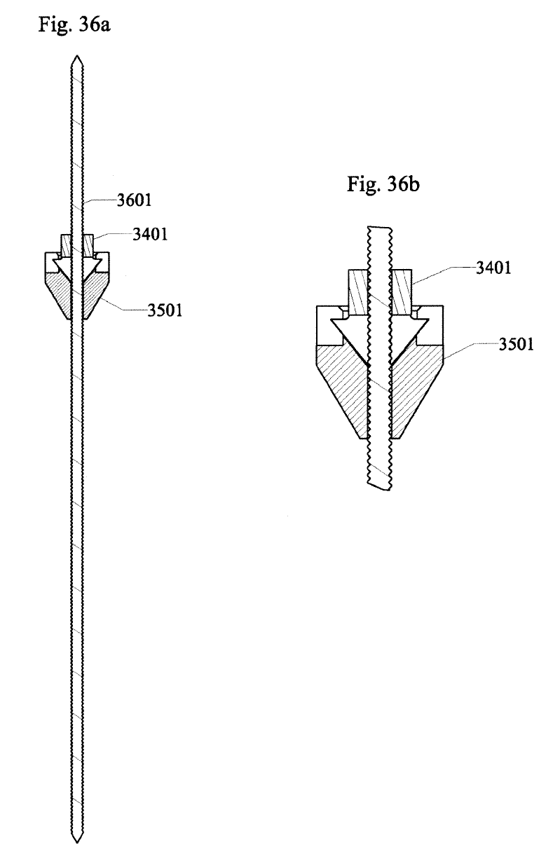

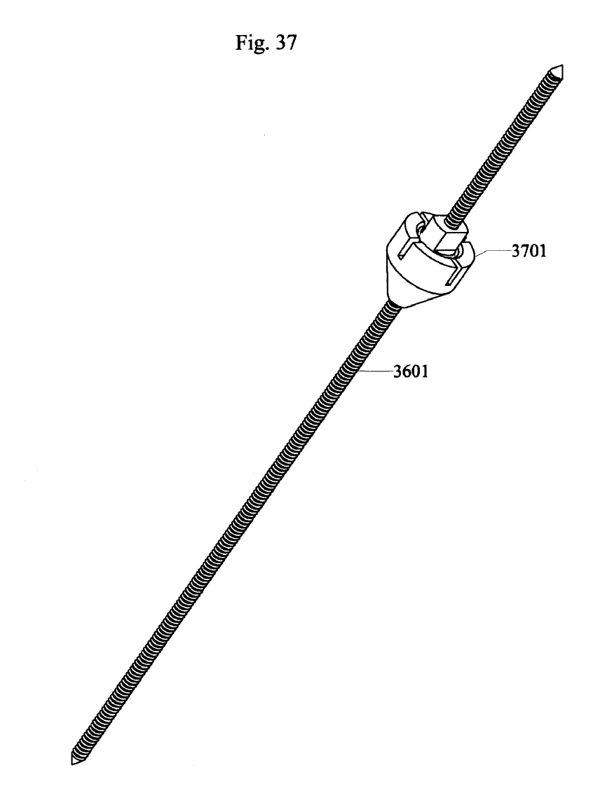

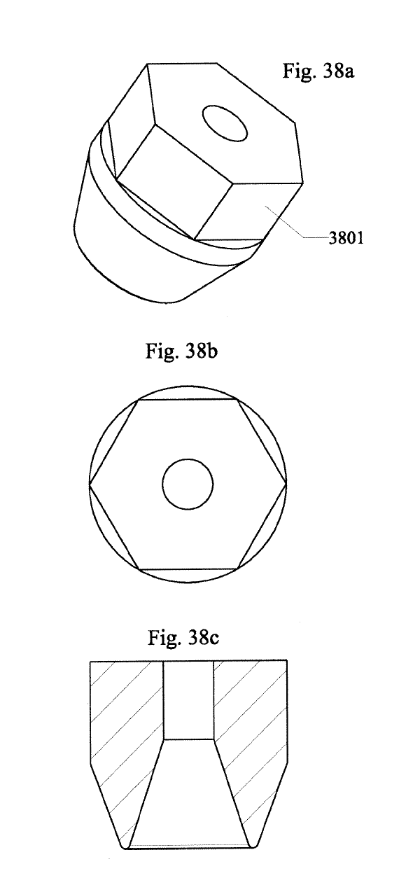

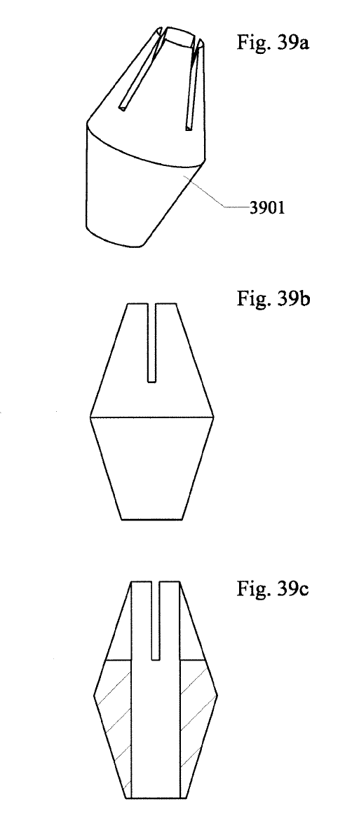

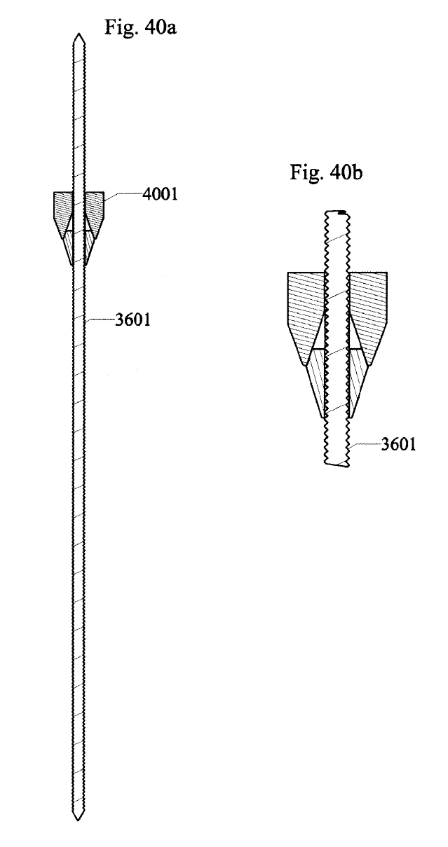

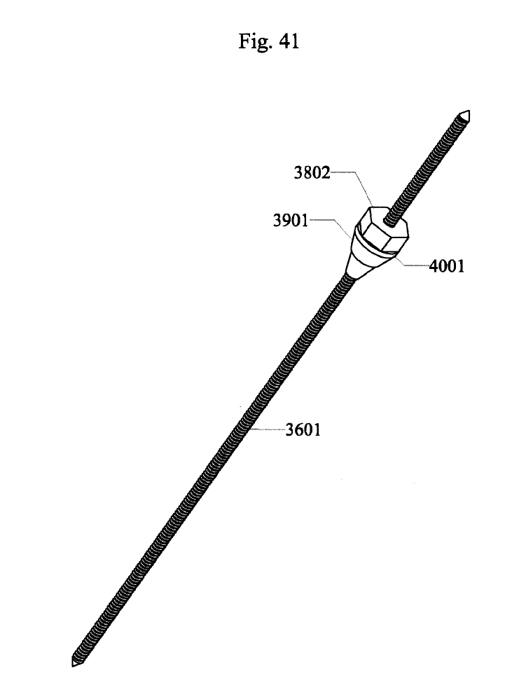

In another embodiment the bone wire compression apparatus 10 is a two part compression device 3401 as shown in FIGS. 34-41. FIGS. 34a-c show various perspectives of the inner component 3401 that might be threaded on its inside wall 3403 that abuts the outer threaded modified bone wire 3601. This inner component 3401 might be sloped inwardly 3403 to nest into an outer component 3501 that could move axially and proximally along the modified bone wire 3601 with the threaded inner component 3401 as depicted in FIGS. 36a and b. The outer component 3501 would not rotate with the inner component 3401 thus preventing twisting or other unnecessary movement against surrounding soft tissue in achieving the desired location on the bone wire 3601.

FIGS. 38-41 illustrate an alternative embodiment of a collet compression device 4001 where the inner non-rotating component 3901 is enclosed by the outer compression device component 3801. Once the collet compression device is in the desired position on the modified bone wire 3601 the user can stop the rotation. In the event the user needs to adjust the compression post procedure the collet compression device 4001 can be advanced distally or proximally along the modified bone wire 3601 by rotating the outer compression device 3801 as appropriate to maintain the desired compression on the treated bone segments.

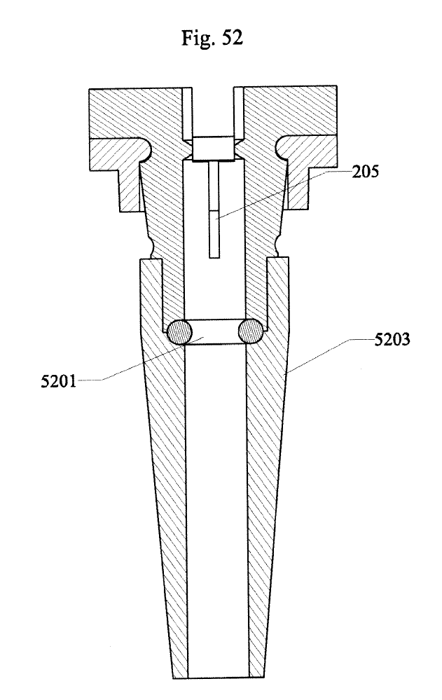

In various embodiments a seal 5201 between a bone wire compression device 5203 as shown in FIG. 52. The seal might be circumferential and would eliminate the ability for solids, liquids and/or gas to pass through the interface between the wire and the compression device 5203. The seal 5201 may be a flexible material that expands around the wire to ensure an ring type of fit ensuring no leakage between the proximal 5205 and distal 5207 portion of the compression device 5203. Any material might be utilized however that would prevent leakage into the proximal portion of the distal end of the compression device. In the preferred use of the seal 5201 it would be located proximal to the end of the relief cuts 205 and distal to the tapered tip 107 of the compression device as shown in FIG. 52.

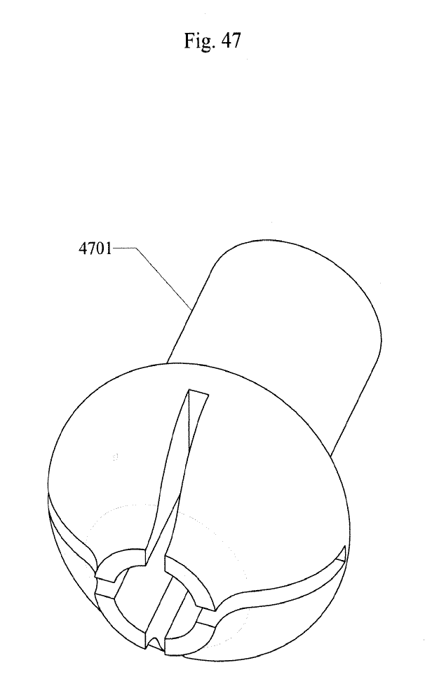

FIG. 47 illustrates an alternative press fit compression device 4701.

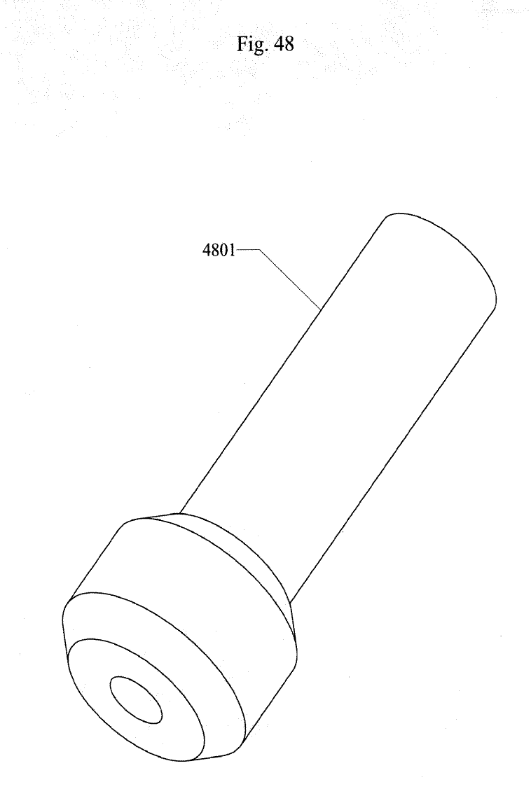

FIG. 48 illustrates an alternative tinnerman clip compression device 4801.

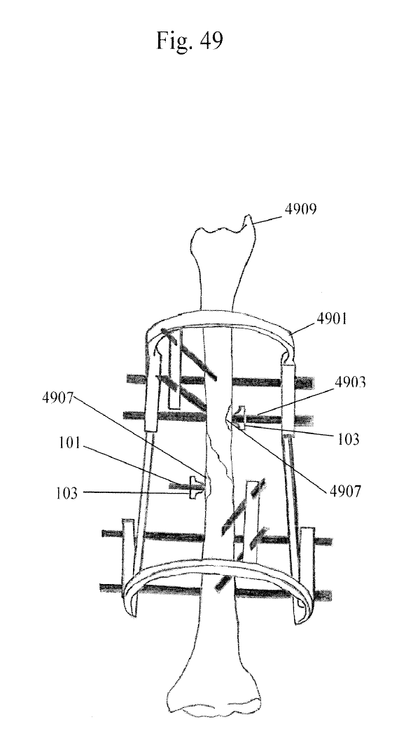

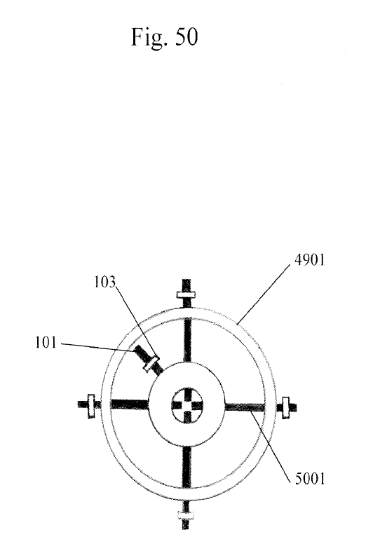

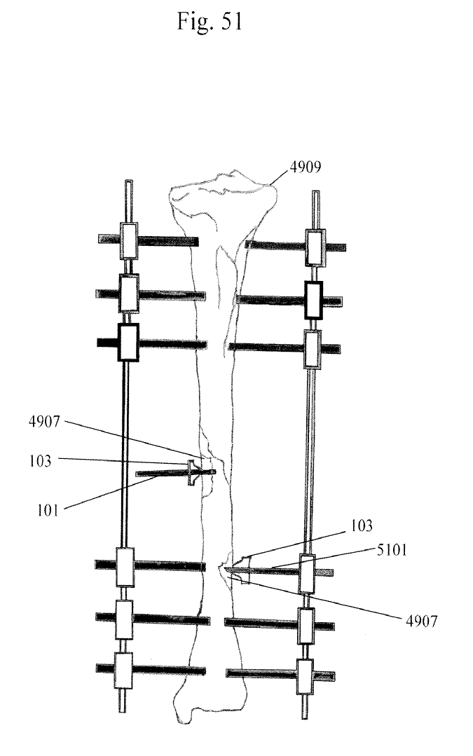

Another example of an alternative use in the treatment of bone trauma and/or deformities with the disclosed compression devices is disclosed in FIGS. 49-51. In these embodiments, the compression devices are utilized with external fixator apparatuses such as the ring external bone fixation apparatus 4901 illustrated in FIG. 49. In that application the compression device 103 might be utilized on a modified pin 4903 or a modified bone wire 4905 to provide compression for bone repair where, for example, the fracture has resulted in a detached bone segment 4907 that needs to be reattached to a main bone 4909. The use of one of the disclosed compression devices, such as, for example, the double recess compression device, in this application would assist the surgeon in obtaining proper placement and compression of the detached segment during the healing process. FIG. 50 illustrates a top view of the ring apparatus 4901 with a compression device 103 on a modified bone wire 101 providing compression independent of the ring pins 5001. FIG. 51 illustrates a compression device utilized on an alternative external fixation device both in conjunction with a modified pin 5101 and a separate modified bone wire 101.

Disclosed are just a few of examples of utilizing the compression devices in alternative bone treatment applications. The disclosed compression devices and modified bone wires, pins, and screws could be utilized anywhere, however that compression is desired for bone and/or tissue treatment due to trauma and/or deformity.

It will be appreciated by persons skilled in the art that numerous variations and/or modifications may be made to the described device as specifically shown here without departing from the spirit or scope of that broader disclosure. The various examples are, therefore, to be considered in all respects as illustrative and not restrictive.

1. A bone compression apparatus, comprising:
an elongate bone wire having a proximal end and a distal end;
a tubular compression device slideably disposed on the bone wire and having a proximal end and a distal end, the tubular compression device defining at least one flexible member; and
at least one male locking tab located on an inside wall of the at least one flexible member of the tubular compression device and radially actuable to selectively engage the bone wire and thereby lock the tubular compression device relative to the bone wire.

2. The apparatus of claim 1, further comprising a pair of opposing relief cuts formed in the distal end of the tubular compression device and extending toward the proximal end of the tubular compression device such that the at least one flexible member of the tubular compression device comprises two flexible halves and the at least one male locking tab includes a pair of male locking tabs located on respective inside walls of the two flexible halves.

3. The apparatus of claim 1, further comprising a locking clip or ring disposed on the tubular compression device, the locking clip or ring applying a radially inwardly-directed circumferential load to the two flexible halves to lock the tubular compression device relative to the bone wire.

4. The apparatus of claim 3, further comprising a locked-position groove formed in the exterior of the tubular compression device adjacent to the distal end of the tubular compression device, and wherein the locking clip or ring is selectively disposed in the locked-position groove to apply the radially inwardly-directed circumferential load to the two flexible halves to force the male locking tabs into engagement with the bone wire.

5. The apparatus of claim 4, further comprising an unlocked-position groove formed in the exterior of the tubular compression device adjacent to the proximal end of the tubular compression device, wherein the locking clip or ring is selectively movable between the unlocked-position groove, to facilitate sliding of the tubular compression device relative to the bone wire, and the locked-position groove, to lock the tubular compression device relative to the bone wire.

6. (canceled)

7. The apparatus of claim 3, wherein the locking clip or ring comprises a C-ring.

8. The apparatus of claim 5, further comprising a helical pathway groove extending between the unlocked-position groove and the locked-position groove to facilitate movement of the locking clip or ring therebetween.

9. (canceled)

10. The apparatus of claim 4, further comprising a pull ring for facilitating movement of the locking clip or ring to the locked-position groove, the pull ring comprising a cylindrical portion slidably disposed on the tubular compression body and a pull flange extending radially outward from an outer wall of the cylindrical portion to be grasped by a user.

11-12. (canceled)

13. The apparatus of claim 1, wherein the bone wire includes a series of female slots disposed adjacent to one another along an axial direction of the bone wire wherein at least one of the female slots is capable of receiving at least one male locking tab.

14-15. (canceled)

16. The apparatus of claim 1, wherein one or more of the male locking tabs is chamfered to facilitate directional sliding of the tubular compression device on the bone wire.

17-30. (canceled)

31. A method of implanting a bone compression apparatus, the method comprising:
screwing a proximal portion of a bone wire into a base bone;
sliding a tubular compression device onto a distal end of the bone wire toward the proximal end, the distal end having a series of female slots disposed adjacent to each other along an axial direction of the bone wire;
positioning the tubular compression device into a desired position on the distal end of the bone wire; and
causing a pair of male locking tabs associated with one of the tubular compression device or a locking clip or ring to be disposed in the female slots of the bone wire to lock the tubular compression device relative to the bone wire.

32. The method of claim 31, further comprising positioning a locking clip or ring on the tubular compression device.

33. The method of claim 32, wherein positioning the locking clip or ring on the tubular compression device comprises moving the locking clip or ring from an unlocked-position groove on the tubular compression device to a locked-position groove on the tubular compression device that is separate and distinct from the unlocked-position groove.

34-35. (canceled)

36. The method of claim 32, wherein the male locking tabs are disposed on an inner wall of the locking clip or ring and positioning the locking clip or ring includes inserting the male locking tabs through receiving slots formed in the tubular compression device and into the female slots of the bone wire.

37-38. (canceled)

39. The apparatus of claim 1, further comprising a cap disposed on the distal end of the tubular compression device applying a radially inwardly-directed circumferential load to the two flexible halves to lock the tubular compression device relative to the bone wire.

40. The apparatus of claim 39, wherein the inner side wall of the cap includes female threads complementary to male threads located on the outside of the distal end of the tubular compression device.

41. The apparatus of claim 1 wherein a seal is located on the inside of the tubular compression device in a location proximal to the relief cuts.

42. The apparatus of claim 41 wherein the seal is an o ring.

43. The apparatus of claim 1 wherein the distal end of the tubular compression device includes a second flexible portion including a pair of female recesses each of which is adapted to receive a respective parallel protruding finger located on the outer distal end of the non-flexible portion of the tubular compression device that, when engaged, lock the at least one male inner locking tab onto the bone wire.

44. The apparatus of claim 1 wherein the distal end of the tubular compression device includes two flexible portions, and wherein each of the male locking tabs is selectively received in a relief on an inner wall of the distal end of the flexible portion of the tubular compression device that, when engaged, locks the at least one male inner locking tab onto the bone wire.
